(19) United States
(12) Patent Application Publication
(10) Pub. No.: US 2025/0136666 A1
(43) Pub. Date: May 1, 2025

(54) A METHOD FOR PRODUCING A LACTOFERRIN POWDER AND USES THEREOF

(71) Applicant: NOUMI LIMITED, Taren Point, New South Wales (AU)

(72) Inventors: Frank BALDI, Taren Point (AU); Sonja KUKULJAN, Taren Point (AU); Tom DICKINS, Taren Point (AU); Ashwantha ENJAPOORI, Taren Point (AU); Bruno TRAZZERA, Taren Point (AU); Christopher BANNING, Taren Point (AU); Nathan BARTLETT, Taren Point (AU); Camille Anne-Genevieve ESNEAU, Taren Point (AU)

(21) Appl. No.: 18/710,869

(22) PCT Filed: Nov. 15, 2022
(86) PCT No.: PCT/AU2022/051362

§ 371 Date: May 16, 2024

(30) Foreign Application Priority Data
Nov. 16, 2021 (AU) 2021903675

(51) Int. Cl. C07K 14/79; A61K 9/00; A61K 9/16; A61K 38/00; A61P 31/14; B01D 15/36; B01D 15/42; B01D 61/14; C07K 1/18; C07K 1/34

(52) U.S. Cl. CPC C07K 14/79; A61K 9/0043; A61K 9/1682; A61P 31/14; B01D 15/361; B01D 15/424; B01D 61/145; C07K 1/18; C07K 1/34; A61K 38/00

(57) ABSTRACT

Disclosed herein is a process for producing a lactoferrin powder. The process comprises extracting lactoferrin from a native lactoferrin-containing substance and concentrating the extracted lactoferrin to produce a liquid concentrate in which the extracted lactoferrin has an improved stability. The liquid concentrate is then pasteurised by heating to a temperature and for a time effective to do so, after which the pasteurized liquid concentrate is dried under non-denaturing conditions to produce a lactoferrin powder in which substantially all of the lactoferrin is native lactoferrin.

### TECHNICAL FIELD

Described herein is a method for producing a lactoferrin powder and uses of such powder.

### BACKGROUND ART

Lactoferrin is a protein found in many animal secretory fluids, including animal milk. Lactoferrin is thought to have numerous health effects or benefits, and is an ingredient in many milk-substitute formulations such as infant formulations.

Lactoferrin can be produced recombinantly or by extracting it from milk or other dairy products such as cheese whey. Typical processes for producing lactoferrin from milk or cheese whey involve an initial pasteurisation step to reduce microbial load, followed by concentration and extraction of the lactoferrin from the milk/whey matrix. The lactoferrin concentrate is then typically freeze dried to produce a particulate substance consisting primarily of lactoferrin. If lactoferrin having a particular particle size and/or particle size distribution are required, then the freeze dried particles can be milled.

It would be advantageous to provide alternative methods for producing lactoferrin.

### SUMMARY OF INVENTION

In a first aspect, the present invention provides a process for producing a lactoferrin powder. The process comprises:
extracting lactoferrin from a native lactoferrin-containing substance and concentrating the extracted lactoferrin to produce a liquid concentrate in which the extracted lactoferrin has an improved stability (i.e. compared to when it was in the lactoferrin-containing substance);
heating the liquid concentrate to a temperature and for a time effective to pasteurize the liquid concentrate; and
drying the pasteurized liquid concentrate under non-denaturing conditions to produce a lactoferrin powder in which substantially all of the lactoferrin is native lactoferrin.

The inventors of the invention the subject of the present application have realised that traditional processes for producing lactoferrin from milk or cheese whey include steps where the conditions experienced by the lactoferrin result in some degree of degradation/denaturing of its protein structure. This causes a consequential decrease in the quantity of recovered lactoferrin and/or quality of the lactoferrin-containing product, in terms of the proportion of native lactoferrin in the product. As would be appreciated, denatured lactoferrin would likely have reduced functionality and biological activity compared with native lactoferrin. Such a decrease in quality went unrecognized in the art for some time because the techniques used to characterize lactoferrin-containing products did not necessarily distinguish sufficiently well between native and non-native forms of lactoferrin, and the products contained sufficient native lactoferrin to have some level of functionality. In the present invention, more lactoferrin can be recovered than is the case for many conventional processes, and the recovered lactoferrin is less denatured than is often the case. In effect, the lactoferrin in the lactoferrin powder of the present invention retains most of its biological activity, despite being presented in a processed and pasteurised form.

Lactoferrin is heat sensitive, like many proteins, and has been found to be particularly prone to degradation if exposed to pH changes at elevated temperatures, as can sometimes occur when liquids such as milk, whey, etc. are heated (especially at and above pasteurisation temperatures). Further, other components of the liquids from which lactoferrin is typically sourced have been found to contribute to the destabilisation of lactoferrin, making it even more susceptible to degradation when heated. The inventors have discovered that a lactoferrin-containing powder in which substantially all (as defined below) of the lactoferrin is native (i.e. un-denatured) lactoferrin can be produced if the lactoferrin presented to the pasteurization stage (in particular, although it would be appreciated that heat may also be applied at other stages of the process) has been extracted from an unpasteurized milk matrix and into liquid concentrate in which the lactoferrin has an improved stability (i.e. when compared to its stability in the native lactoferrin-containing substance, such as milk).

In this manner the lactoferrin is not exposed to such destabilising effects that can occur when liquids such as milk are heated during pasteurization (which is essential for regulatory compliance with many industrial standards for food products), and the process of the present invention has been found to be less damaging to the structure of the lactoferrin protein as is the case for conventional processes. Indeed, the inventors observe that pasteurization of milk results in losses of lactoferrin recovery of around 25%, and that there may be some other conformational changes to lactoferrin when pasteurized in a milk matrix. Furthermore, in the present invention, the lactoferrin-containing powder is produced in a minimum number of process steps, and does not include steps routinely used to produce lactoferrin powders such as evaporation, etc. The inventors recognise that every additional process step can damage the lactoferrin and/or result in a lower overall recovery.

In some embodiments, the extracted lactoferrin may have an improved stability because of the absence in the liquid concentrate of species that destabilise the lactoferrin. Species typically found in a milk matrix, for example, include proteins such as casein and whey proteins, lipopolysaccharides, sugars and ionic species such as calcium and magnesium ions, some or all of which might deleteriously affect the stability of lactoferrin in a milk matrix (either directly or indirectly, e.g. by causing a pH shift). Pasteurizing a lactoferrin-containing liquid concentrate which does not contain such potentially deleterious species means that the lactoferrin is not being heated in the presence of species which might have a destabilising effect on the lactoferrin.

In some embodiments, the extracted lactoferrin may have an improved stability because of the presence in the liquid concentrate of species that stabilise the lactoferrin. The liquid concentrate may, for example, include one or more of the following: a pH adjusting agent, a stabilising mineral and a dissolved gas. Specific examples of such species, and the inventors' understanding of their protective mechanisms, will be described below.

In some embodiments, the native lactoferrin-containing substance may be milk, preferably skim milk. Cold bowl fat separation has been found to be advantageous for producing skim milk because it does not involve subjecting the lactoferrin to even mildly elevated temperatures whilst it is still contained within the milk matrix. As described in further detail below, lactoferrin can easily be denatured at this initial step.

In some embodiments, the lactoferrin may be extracted from the native lactoferrin-containing substance using ion exchange chromatography. The lactoferrin may be eluted from the ion exchange column separately to the other components of the substance (e.g. milk matrix).

In some embodiments, the extracted lactoferrin may be concentrated using ultrafiltration, the lactoferrin being contained in the filtrate, for subsequent reconstitution in the liquid concentrate.

In some embodiments, the pasteurized liquid concentrate may be cooled immediately after pasteurisation, thereby reducing to an absolute minimum the length of time the lactoferrin spends at elevated temperatures (whilst still complying with regulatory requirements regarding pasteurisation).

In some embodiments, the pasteurized liquid concentrate may be dried under non-denaturing conditions to produce a lactoferrin-containing product by spray drying. Spray drying (particularly multi-stage spray drying) has advantageously been found by the inventors to reduce the necessity for steps such as evaporation, which necessarily involve the application of potentially damaging amounts of heat before further heat application and/or processing during the final powder forming stage. Particular advantages may be achieved if multiple stage spray drying is used, with parameters such as the nozzle size, inlet and outlet temperatures and residence times being used to advantage in order to even further reduce cumulative applied temperatures and hence contribute to producing substantially native lactoferrin-containing powders having specific properties, such as a specific desired particle size and/or particle size distribution.

In some embodiments, the lactoferrin powder may consist essentially of lactoferrin. In alternative embodiments, up to about 10% w/w (preferably less than about 5% w/w) of other substances may be present in the lactoferrin powder, such being inevitable by-products of the process of the present invention. For example, moisture and other non-specific-proteins may be contained in the final product, provided that their presence does not deleteriously affect the product's utility.

In a second aspect, the present invention provides a lactoferrin powder produced by the process of the first aspect of the present invention.

In a third aspect, the present invention provides a food product comprising or consisting essentially of the lactoferrin powder produced by the process of the first aspect of the present invention.

The inventors have also discovered that the highly functional and biologically active lactoferrin contained in the lactoferrin powders produced in accordance with the process of the first aspect of the present invention may have therapeutic activity or an enhanced therapeutic activity because of the unique properties of the lactoferrin powder. Specifically, experiments conducted by the inventors (described below) have demonstrated that powdered lactoferrin produced in accordance with the present invention has an inhibitory effect on virus replication in vitro and in vivo. The inventors believe that the results of their preliminary experiments lead to a reasonable prediction of the therapeutic applications disclosed herein. Further experiments, both currently underway and planned, are expected to confirm the inventors' predictions.

In a fourth aspect, therefore, the present invention provides a nasal spray comprising the lactoferrin powder produced by the process of the first aspect of the present invention.

In a fifth aspect, the present invention provides a pharmaceutical composition comprising the lactoferrin powder produced by the process of the first aspect of the present invention and a pharmaceutically acceptable excipient.

In a sixth aspect, the present invention provides a method for preventing or treating a viral infection in a patient, the method comprising administering to the patient (e.g. via the patient's airways) a formulation comprising the lactoferrin powder produced by the process of the first aspect of the present invention or the pharmaceutical composition of the fifth aspect of the present invention.

In a seventh aspect, the present invention provides a method for preventing or treating a viral infection in a patient, the method comprising nasally administering to the patient a formulation comprising the lactoferrin powder produced by the process of the first aspect of the present invention or the pharmaceutical composition of the fifth aspect of the present invention.

In an eighth aspect, the present invention provides the lactoferrin powder produced by the process of the first aspect of the present invention for use as a medicament.

In a ninth aspect, the present invention provides the lactoferrin powder produced by the process of the first aspect of the present invention for use in preventing or treating a viral infection in a patient.

In a tenth aspect, the present invention provides the use of the lactoferrin powder produced by the process of the first aspect of the present invention for the manufacture of a medicament for preventing or treating a viral infection in a patient.

The inventors also note that lactoferrin has exhibited immunomodulatory and anti-inflammatory activities. The inventors' note that the lactoferrin powder produced by the process of the first aspect of the present invention may provide for an improved delivery form of lactoferrin for the treatment and prophylaxis of conditions relating to such activities.

Other aspects, features and advantages of the present invention will be described below.

### BRIEF DESCRIPTION OF DRAWINGS

Embodiments of the present invention will be described in further detail below with reference to the following drawings, in which:

FIG. 1 is a block flow diagram of an embodiment of the first aspect of the present invention;

FIG. 2 is a graph showing the viral load for a preliminary BEC study using cells from n=1 asthmatic patient. Differentiated primary human bronchial epithelial cells with OC43 viral load following treatment with lactoferrin at 100 μg/mL (LF100▪) or 1000 μg/mL (LF1000 ▴);

FIG. 3 is a graph showing the viral load for another preliminary study using n=2 healthy donors (n=4 replicates each=8 total), untreated (•) and 1000 μg/mL (LF1000 ▴);

FIG. 4 is a graph showing the number of infectious virus over time for 2× healthy donors (n=4 replicates each=8 total), untreated (•), 100 μg/mL (LF100▪) and 1000 μg/mL (LF1000 ▴);

FIG. 5 is a graph showing the number of infectious virus over time for n=1 healthy donor (n=3 replicates each=6 total, lactoferrin was replenished at 48 h), untreated (·) and 100 μg/mL (LF1000

FIG. 6 depicts the experimental plans for (A) in vivo study 1&2 and (B) in vivo study 3;

FIG. 7A is a graph showing OC43 viral load in nasal turbinate in untreated (vehicle) mice, versus mice treated with 1.5 ug of lactoferrin intranasally over a 48-hour time course;

FIG. 7B is a graph showing OC43 infectious virus measured in MRC-5 cells using TCID--50 assay in nasal wash samples;

FIG. 8A is a graph showing OC43 viral load in nasal turbinate in untreated (vehicle) mice, versus mice treated with 1.5 ug of lactoferrin intranasally over a 48-hour time course;

FIG. 8B is a graph showing OC43 infectious virus measured in MRC-5 cells using TCID--50 assay in nasal wash samples;

FIG. 9 is a graph showing A) IFN-β and B) IFN-22/3 levels between vehicle and lactoferrin treated mice as assessed by qPCR; and

FIG. 10 is graphs showing (A) Macrophages, (B) Neutrophils and (C) Lymphocytes in nasal turbinate in untreated (vehicle) mice, versus mice treated with 150 ug of lactoferrin at the time of infection and twice daily thereafter and BAL.

### DESCRIPTION OF EMBODIMENTS

As described above, the present invention provides a process for producing a lactoferrin powder with what the inventors believe has a unique property in that the lactoferrin in the powder composition retains most of its biological activity, despite being having been pasteurised and processed into a powder. The process comprises extracting lactoferrin from a native lactoferrin-containing substance and concentrating the extracted lactoferrin to produce a liquid concentrate in which the extracted lactoferrin has an improved stability compared to when it was in the lactoferrin-containing substance (as evidenced by the lactoferrin in the powder having substantially retained its biological and functional properties, as well as its % recovery, described below). The liquid concentrate is then pasteurised by heating to a temperature and for a time effective to do so, after which the pasteurized liquid concentrate is dried under non-denaturing conditions to produce a lactoferrin powder in which substantially all of the lactoferrin is native lactoferrin.

The so-produced lactoferrin powder has an improved biological activity over many other lactoferrin-containing powders due to its higher proportion of native lactoferrin. The functional properties of the lactoferrin powder are also advantageous (e.g. due to its solubility, blendability specific particle size attributes and morphology, etc.). The process of the present invention can also result in improved yields of native lactoferrin, when compared with processes where lactoferrin extraction occurs post-pasteurisation.

The present invention results in the production of a lactoferrin powder in which substantially all of the lactoferrin is native lactoferrin. Native lactoferrin is not denatured or only negligibly changed, with the lactoferrin protein retaining its structure and hence its biological activity and health effects. As will be appreciated, it is possible that not all of the lactoferrin which is processed in accordance with the present invention will be completely un-denatured. A small degree of denaturation may be inevitable. The lactoferrin powder may, for example, contain greater than 90% native lactoferrin, greater than 95% native lactoferrin or greater than 98% native lactoferrin. The lactoferrin powder may, for example, contain about 90%, 91%, 92%, 93%, 94%, 95%, 96%, 97%, 98%, 99% or even 100% native lactoferrin.

The lactoferrin powder produced by the present invention may consist essentially of lactoferrin (i.e. with the exception of small amounts of inevitable impurities, by-products, degradation products, etc. the entity of the product is lactoferrin). Such a high level of purity may not be required, however, and, in some embodiments (e.g. where a cheaper product is commercially attractive) it may be sufficient for up to about 10% w/w of other components to be present in the lactoferrin powder. Such other components may include inevitable by-products of the process of the present invention, but may also include additives provided for functional reasons (e.g. anti-caking agents, preservatives, etc.).

The lactoferrin powder produced by the present invention may have any appropriate particle size, or range of particle sizes, and this will be described in further detail below in the context of the drying step. By way of example, one lactoferrin-containing powder produced in accordance with the present invention and characterised by the inventors as a fine powder has a particle size distribution D50=38.2 micron and D90=72.3 micron. Another lactoferrin-containing powder produced in accordance with the present invention and characterised by the inventors as an intermediate powder has a particle size distribution D50=46.2 micron and D90=98.7 micron. Yet another lactoferrin-containing powder produced in accordance with the present invention and characterised by the inventors as a coarse powder has a particle size distribution D50=56 micron and D90=130 micron.

The proportion of native lactoferrin in any given sample of a lactoferrin-containing powder can be determined as described below, in conjunction with published techniques. Generally speaking, native lactoferrin protein has a molecular weight of around 80 kDa, has around 10-20% iron saturation and is salmon pink in colour. Native lactoferrin powder will also have a comparable bioactivity to that of its source, whilst the biological activity of damaged lactoferrin will be compromised.

An assay which the inventors have used to determine lactoferrin purity in lactoferrin powders (e.g. the purity percentage of lactoferrin vs other protein, in a sample), but which they believe is not suitable for quantification or for discriminating between native (i.e. non-denatured) and denatured forms of lactoferrin is the Chinese GB RP-HPLC (GB 1903.17-2016) method. This method, implemented by the Standards Administration of China for testing bovine lactoferrin powders imported into China, involves the injection of a high concentration of protein (50 μl of 10 mg/ml sample (0.5 mg per injection)), which was close to the upper end of the column binding capacity. Due to this some significant differences were observed in bovine lactoferrin determination.

An assay which the inventors have used to determine the lactoferrin quantity (as a percentage) in finished powders is the Callaghan Innovation RP-HPLC method, in which native lactoferrin is distinguishable from denatured lactoferrin because only native lactoferrin binds to the HPLC column. The Callaghan Innovation method is described in J. Billakanti et. el., International Dairy Journal 99 (2019) 104546 and is conducted using an Aeris™ 3.6 μm WIDEPORE XB-C8 200 Å analytical LC column (Part-OOG-4481-EO, 250 [1] 4.6 mm), a C8 Security Guard Ultra Holder (Part-AJO-9000) and Security Guard Ultra Cartridges (Part-AJO-8771) and detection is carried out at a wavelength of 280 nm. The method uses w/w preparation of the samples, rather than the v/v based sample preparation used in other protocols, which gives reproducible and reliable results over time and, as such, a more accurate analysis.

The inventors have found that a combination of such assays enables both a lactoferrin quantity (i.e. percentage recovery) and quality (i.e. proportion of undenatured lactoferrin) of a particular lactoferrin-containing powder to be determined. As noted above, the invention the subject of this application relates, at least in part, to the realisation that both of these assays are required in order to adequately characterise native-lactoferrin containing powders.

One of the key features of the present invention is that pasteurisation is carried out only after the lactoferrin has been extracted from the native lactoferrin-containing substance and concentrated. Lactoferrin in the liquid concentrate has an improved stability compared to when it was in the native lactoferrin-containing substance, and is therefore less susceptible to denaturation caused by the temperatures that must be applied in order to pasteurise the product. Pasteurisation conditions (minimum temperature, time, etc.) must be met in order to meet regulatory (and safety) requirements but, in the present invention, the stability of the lactoferrin to the applied heat is greater than it would have been if pasteurisation were carried out whilst the lactoferrin was contained in the native lactoferrin-containing substance. In this manner, substantially all of the lactoferrin in the liquid concentrate can remain in its native form. In contrast, pasteurisation of milk, for example, denatures up to a 25% native lactoferrin in the milk, resulting in a corresponding decrease in lactoferrin recovery at the outset.

In the context of the present invention, the feature “lactoferrin having an improved stability”, or the like, is to be understood to mean that the stability of lactoferrin in the liquid concentrate is greater than the stability of lactoferrin in the native lactoferrin-containing substance. Routine testing (including that described below) can be used to assess the relative stability of lactoferrin in different carriers.

Lactoferrin can undergo either or both of thermal and chemical (e.g. because a shift in pH causes denaturation) degradation, unless it is protected in the manner described herein. The lactoferrin may have an improved heat stability because of the absence in the liquid concentrate of species that destabilise the lactoferrin. Alternatively, or in addition, the lactoferrin may have an improved heat stability because of the presence in the liquid concentrate of species that stabilise the lactoferrin.

As described above, species typically found in dairy products such as whey and milk include proteins such as casein and whey proteins, sugars (e.g., lactose), fats (e.g. lipopolysaccharides) and ionic species such as calcium and magnesium ions. Any or all of these species may contribute to the relative lack of heat-stability of lactoferrin, either directly (e.g. by denaturing or otherwise reacting with the lactoferrin upon heating) or indirectly (e.g. by causing a pH shift which destabilises the lactoferrin at higher temperatures). Isolating the lactoferrin from the matrix of the native lactoferrin-containing substance prevents (or at least reduces) any such deleterious interactions from occurring.

The lactoferrin-containing liquid concentrate may include additional species which can help to stabilise the lactoferrin as it is heated. Such species would need to be compatible with the end use of the lactoferrin powder (e.g. be GRAS, compatible with a food product, etc.) and otherwise not deleteriously affect the performance of the invention. These species may either be added to the liquid concentrate or may already be present in the concentrate (e.g. in the eluant, for example).

In some embodiments, the liquid concentrate may, for example, include processing aids such as one or more of the following: a pH adjusting agent, a stabilising mineral and a dissolved gas. Such species may be added to the liquid concentrate via a carrier liquid/gas or directly in their present form pre-pasteurisation.

The conductivity of the liquid concentrate (which is a measure of the concentrate's ability to pass or carry an electric current) may, for example, be adjusted by manipulating the amount of minerals such as sodium chloride, or those found in town water supplies, in the concentrate. The present invention can maintain the native aspect of the lactoferrin by controlling conductivity to further stabilise the lactoferrin concentrate prior to pasteurisation. The liquid concentrate may be RO water, although water having a small mineral content (e.g. town water) may not adversely affect the process and may help to reduce the overall cost of the process. Indeed, water containing low levels of minerals may also act to further stabilise the lactoferrin.

The pH of the liquid concentrate may be controlled in a manner that prevents the generation of unwanted contaminants or in order to meet importing country requirements or customer specifications. Generally speaking, for example, the pH of lactoferrin needs to be compliant with a range of between 5.2-7.2 in order to comply with regulatory requirements. pH adjustment of the liquid concentrate may be used in two instances, firstly to stabilise the lactoferrin for pasteurisation or secondly to ensure final conformance to standard.

The inventors note that apo-lactoferrin (non-iron bound) is more easily damaged than holo-lactoferrin (iron bound), which is much more stable. Thus, conditions in the liquid concentrate that favour the formation of holo-lactoferrin may improve the lactoferrin's heat resistance.

The use of certain acids to lower the pH may be appropriate as such may help to reduce the impact of denaturation. However, doing so may increase contaminant levels or negatively impact sensory characteristics in the concentrate, which is undesirable and may not be permitted in lactoferrin-containing products in some markets.

The use of sodium bicarbonate (or other bicarbonate) has also been found to stabilise lactoferrin during pasteurisation, possibly due to interactions between the bicarbonate ion and the iron binding sites at the C and N lobes of the lactoferrin molecule. Such may also help to manage the pH of the end product, so that it sits at a pH between 5.2-7.2 (preferably 5.7), as required for food products/additives.

It is within the ability of a person skilled in the art, using no more than the information disclosed herein and routine trial and experimentation to determine whether any particular species can be used in the method of the present invention.

As noted above, additional components may need to be added during the process for the purpose of meeting regulatory requirements. For example, food products are required to have a pH of greater than 5.2 and, in some embodiments, it may be necessary to add a pH adjusting agent post-pasteurisation in order for the final product of the invention to be compliant. It is within the ability of a person skilled in the art to determine the need for, and to select such additional components, using the teachings contained herein and no more than routine testing.

The inventors also note that additional processing steps might be used instead of adding further components in order to achieve the same effect. For example, anion exchange can be used to adjust the pH of a sample and it may sometimes be advantageous to perform anion exchange instead of adding further substances in order to adjust the pH of the sample.

The lactoferrin may be extracted from any native lactoferrin-containing substance. Typically, the native lactoferrin-containing substance is milk, this being in plentiful supply and not contaminated with any processing aids (as would be the case, for example, with whey derived from cheese making processes). Skim milk is preferred because fat containing substances can negatively impact the lactoferrin binding capacity of the ion exchange process. Fat level should ideally be maintained below 0.1%.

In embodiments where the native lactoferrin-containing substance is skim milk, the skim milk may be produced using a cold bowl fat separation process, such a process avoiding the elevated temperatures of hot bowl fat separation. Whilst such temperatures are only around 50° C. (significantly lower than pasteurisation temperatures), any heating of lactoferrin whilst it remains in the milk matrix may be best avoided, lest it damage even some of the lactoferrin protein. Furthermore, bacterial growth is lower at low temperatures, which provides improved milk quality over hot bowl separation.

In the present invention, lactoferrin is extracted from a native lactoferrin-containing substance. Any suitable extraction technique may be used for this step, provided that it is compatible with the outcomes of the present invention. One suitable technique is for the lactoferrin to be extracted from the native lactoferrin-containing substance using cation ion exchange chromatography.

Generally speaking, ion exchange chromatography involves passing the native lactoferrin-containing substance (e.g. skim milk) through a column. The lactoferrin binds to and is retained on the column, whilst the remaining components of the milk matrix pass through the column. The retained lactoferrin can subsequently be eluted using a different solvent, such as a sodium chloride eluant. In a specific embodiment, such a process may utilise SP Sepharose Big Bead resin composed of large, crosslinked agarose beads (100-300 μm) modified with sulphonate (SP) strong cation exchange groups. During this process, the positively charged lactoferrin binds to the negatively charged resin.

In the present invention, the extracted lactoferrin is concentrated to produce a liquid concentrate in which the lactoferrin has an improved heat stability. Again, any suitable technique may be used for this step, provided that it is compatible with the outcomes of the present invention. One suitable technique for concentrating lactoferrin is ultrafiltration.

Generally speaking, ultrafiltration involves concentrating the lactoferrin in a selected liquid carrier (e.g. RO water) whilst removing other contaminants (e.g. the eluant from the extraction step). In a specific embodiment, ultrafiltration membranes filter media designed to retain proteins with specific molecular weight (above 10-50 kDa) are used to concentrate the lactoferrin.

The amount of lactoferrin in the liquid concentrate pre-pasteurisation may, for example, be between about 9 to 12 wt % (e.g. at about 11% solids). This amount of lactoferrin is higher than is often present in conventional processes, which can range from 6-8 wt % lactoferrin. Advantageously, reconcentrating at the time this occurs in the process of the present invention results in a higher proportion of lactoferrin in the concentrate, which the inventors have found can enable the concentrate to be dried (e.g. by spray drying) to produce a powder having better particle size flexibility without adding another further concentration process.

In the present invention, the liquid concentrate is pasteurised by heating to a temperature and for a time effective to do so. Pasteurisation is a well-practiced technique and appropriate pasteurization conditions for a given sample can readily be ascertained by persons skilled in the art. An effective pasteurisation can be achieved by heating the lactoferrin-containing liquid concentrate to a minimum temperature of 72° C. for a minimum time of 15 seconds. Other forms of pasteurisation are known in the art, and it is expected that the advantageous effects of the present invention may apply to such as well.

Finally, the pasteurized liquid concentrate is dried under non-denaturing conditions to produce a lactoferrin powder in which substantially all of the lactoferrin is native lactoferrin. Conventional drying techniques include freeze drying and spray drying, with spray drying being preferred in the present invention because it is generally better for producing particles having a relatively homogeneous particle size and it is not usually necessary to mill the product, as is sometimes the case with freeze drying. Freeze dried powders can sometimes also lack functionality, for example, they may have a coarser particle size and not readily blend into milk powders.

Any suitable spray drying technique and apparatus may be used. Typically, spray drying apparatus allow for the manipulation of parameters including nozzle size, inlet and outlet temperatures and residence time, all of which can be adjusted to produce a powder having desired physical properties. The inventors note that the physical properties of the resultant powder also directly influence its functional properties, such as its mixability with aqueous liquids and flowability.

Multiple stage spray drying may advantageously be used in the present invention because it enables, amongst other advantages, lower nozzle temperatures to be applied in spraying and drying the lactoferrin powders. As described above, the less time the lactoferrin is exposed to relatively higher temperatures, the better.

For example, using a single stage dryer, the outlet temperature governs the exit powder temperature. A multi-stage dryer, however, has the ability to manipulate the exit powder temperature, using secondary heating/cooling (secondary temperature manipulation is achieved via a fluid bed—the fluid bed has its own heating and cooling sources). The secondary drying system allows for less aggressive drying temperatures in the outlet, because the secondary drying system can compensate and complete drying at lower temperatures, thereby preserving the structure of lactoferrin. The inventors note that such spray drying may also contribute to the improved health effects/functional benefits of the lactoferrin in the powder, and that the cumulative impact of all of the steps in the process of the present invention may lead to the powder having an improved functional lactoferrin.

It is within the ability of a person skilled in the art to adjust the various parameters of a spray dryer in order to not denature the lactoferrin and to produce a powder having the desired functional properties. Specific examples of two trials carried out by the inventors in this regard will be described below.

### Nozzle Size Trials

The inventors have found that the combination of various nozzle sizes, targeted nozzle pressures and use of a multi-stage spray dryer allows for the production of lactoferrin powders with specific and unique particle size distributions.

As described below, two example particle size distributions were targeted, each of which is associated with specific functional properties in the finished product, namely mixability (powder and/or aqueous) and powder flowability. Particular elements of the spray dryer operation were varied, including: (1) the spray nozzle swirl chamber and orifice size combination; (2) the main chamber's inlet and outlet temperature; (3) the nozzle pressure; (4) the degree of agglomeration, dictated partially by the point of re-entry of fine particulates in the process; (5) and the concentration of solids in the feed stream.

During these nozzle size trials, powder performance testing was carried out in real time and parameters were adjusted in real time to achieve the advantageous particle size distribution (i.e. the finer and coarser variants as described below). This allows modifications to the running conditions during the trial, thus instantly validating the process.

The conclusion of the trials produced two main variants, a finer powder and a coarser variant. The finer powder is characterised by a D50 value of approximately 35-40 μm and d90 value of approximately 50-55 μm, while the coarser variant is characterised by a D50 value of approximately 50-60 μm and a D90 value of 125-130 μm. The targeted aqueous mixability and powder flowability characteristics required in the powder were achieved.

| | 0.58 Nozzle-Coarser powder |
|---|---|
| 0.34 Nozzle-Finer Powder 0.58 Nozzle-Coarse powder | 0.34 Nozzle-Fine Powder |
| The air temperature at the dryer inlet | The air temperature at the dryer inlet |
| is targeted at approximately 180- | is targeted at approximately 175- |
| 190° C. The lactoferrin concentrate | 185° C. The lactoferrin concentrate |
| does not reach these high | does not reach these high |
| temperatures, but is instead exposed | temperatures, but is instead exposed |
| to drying for a short residence time | to drying for a short residence time |
| (<2 minutes), where powder | (<2 minutes). The powder |
| temperature is typically maintained | temperature is typically maintained |
| at 65° C. and never greater than 75° C. | at 65° C. and never greater than 75° C. |
| The exit temperature from the | The exit temperature from the |
| process is less than 40° C. | process is less than 40° C. The |
| The nozzle pressure during spray | powder residence time is generally |
| drying is approximately 150-160 | maintained with variations in the |
| bar, generating a finer particle size. | concentrate feed flow rate. |
| The variant utilises a smaller nozzle | The nozzle pressure during spray |
| orifice size, generally 0.34 THOU | drying is approximately 100-110 |
| (thousandth of an inch). | bar, generating a larger particle size. |
| | The variant utilises a larger nozzle |
| | orifice size, generally 0.55 THOU. |

### Multi-stage Spray Dryer

Use of a multi-stage spray dryer (MSD) can greatly assist in the preservation of the native structures of the lactoferrin powder, by removing water via heated air through three stages; (1) the main drying chamber; (2) the static fluidised bed and (3) the vibrating fluidised bed. In contrast to single-stage dryers, the MSD allows for further water removal after stage 1, via the stage 2 and 3 processes. These processes permit the further removal of water to occur at lower temperatures where the water activity is already reduced and the lactoferrin is susceptible to damage from higher temperatures. The static fluidised bed (stage 2) temperature is never greater than 75° C.

In contrast to a MSD, when utilising single-stage dryers it is not possible to match the temperature profile of drying. Single-stage dryers require the utilisation of either higher drying temperatures, or longer residence times to achieve a similar outcome. This results in an increased exposure to heat when utilising single-stage dryers and increased damage to the native structure of the lactoferrin powder.

### Percentage Solids in Lactoferrin Concentrate Trials

A feed stream into the spray dryer with a lower concentration of solids was also trialled. The upstream ultrafiltration plant concentrated the lactoferrin concentrate to 10-12% solids before it was pasteurised and supplied to the spray dryer.

A modified heating profile and spray pattern was applied to the feed stream, which allowed the above defined mixability and flowability, and the particle size distribution, to be maintained, whilst improving lactoferrin quantification. Typically, lowering the feed stream per centage solids will impact the particle size distribution of the powder, decreasing the average particle size and shifting the particle size distribution down in size. Manipulation of other process parameters, including: (1) the nozzle swirl chamber; (2) the nozzle orifice size; and (3) nozzle pressure, to maintain the particle size distribution when spray drying a lower per centage solids feed stream, was found to result in an increased lactoferrin recovery.

Pasteurisation and spray drying was completed at the same concentration of solids, namely 10-12%. In contrast, prior art processes of which the inventors are aware typically pasteurise at lower solids (7-8%), apply evaporative concentration techniques and then spray dry at higher per centage solids. This permits a higher per centage recovery to be achieved, in line with the teachings in the published literature, but with the adverse consequences described above on the structural and biological functionality of the recovered lactoferrin. Thus, whilst other processes may achieve greater recovery of lactoferrin than the process of the present invention by pasteurising at lower solid concentrations, the present invention can enable the same percentage recovery by using higher percent solids before drying concentrate.

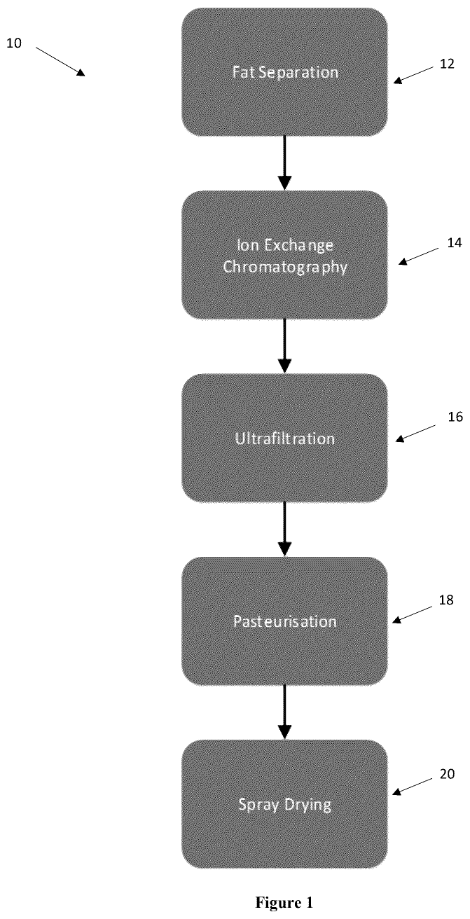

A specific embodiment of the process of the present invention 10 will now be described in detail with reference to the flowchart shown in FIG. 1.

### 1) Milk Delivery/Handling—to Site

Whole Milk is collected from farms using a double skinned road tanker. At the point of pick up, the milk temperature is verified to ensure it meets the regulatory requirements. The milk is delivered to site and unloaded and stored in Chilled silos to temperature <5 degrees Celsius. Age of the milk is monitored to ensure it is processed withing 24-48 hours of receival to site. Milk temperature is monitored, and milk quality is managed via testing of the milk for acidity levels for each tanker load that is delivered

### 2) Fat Separation (Cold Bowl Skimming) 12

The Separation of milk is an important process in a milk treatment plant. At this step, whole milk is separated into the cream and skim portions for further processing. Traditional separation involves the use of hot bowl hermetic separators which require the milk to be heated to 50° C. during the separation processing, which encourages bacterial growth. In the process of the present invention, however, Cold Bowl milk separation technology is preferred, which allows effective separation of the cream from the skim portion at temperatures of 24-26° C., ensuring the milk quality is maintained and minimising bacterial growth during processing.

### 3) Skim Pre-Treatment (Clarification/Filtration)

The skimmed milk with a fat content of <0.1% is transferred to the lactoferrin process where it undergoes clarification and filtration. Clarification is conducted utilising a GEA Bacterial Removal System (BRS), a process designed to reduce the bacterial loading of skimmed milk during processing. Bacteria of concern includes spore formers such as Bacillus cereus, heat-resistant up to 128° C. and cold-tolerant at the same time, which can negatively impact the shelf life of the milk for upstream processes. The milk is also filtered to ensure that excessive fat and other impurities are removed prior to the ion exchange (i.e. lactoferrin removal) process in the columns.

### 4) Ion Exchange Process 14

Extraction of the lactoferrin from the skim milk is conducted through an ion exchange process where the skim milk is passed through columns (Holding Vessels) which contain specific SP Sepharose Big Bead resin. The resin is composed of large, crosslinked agarose beads (100-300 μm) modified with sulphonate (SP) strong cation exchange groups. It is designed for industrial applications and the large particle size and physical stability of the base matrix allow the resin to be utilised in high volume commercial extraction processes. The ion exchange capability of the resin binds the lactoferrin from the skim milk for collection through the elution process. The ion exchange process is a batch process that requires the columns be loaded with lactoferrin until the loading efficiency falls below 93%. At this stage, a co-elution process is used to extract the lactoferrin.

### 5) Eluate Collections and Storage

On completion of the column loading process, the lactoferrin is washed off the resin using specifically prepared buffer solutions, which is a two-step elution process. The first part of the elution process uses a lower buffer concentration that is designed to remove undesirable protein impurities, which consists mostly of Lactoperoxidase, but other impurities such as whey proteins and minerals will also be removed at this point. Step two of the elution process uses a higher buffer strength to strip off the lactoferrin from the Sepharose big beads and is collected into a storage vessel for further processing. Buffer conductivity levels are controlled to ensure impurities are removed, which impacts the final efficacy levels (i.e. content and purity) of the lactoferrin product.

### 6) Ultrafiltration 16

Accumulation of multiple elutions is a prerequisite for the ultrafiltration process. This step in the process is specifically designed to remove the buffer reagents from the lactoferrin until the desired conductivity level is reached in the lactoferrin solution. It is designed to remove impurities like sodium, chloride and nitrates. The filtration process will then continue to achieve final concentration of the product for pasteurisation. Total solids and pH of the concentrate at the completion of this step is carefully managed in order to maintain the native lactoferrin structure. Total solids and pH may be adjusted using the process water, and other pH adjustment additives may be used at this point to ensure the concentrate is within required tolerance levels.

### 7) Pasteurisation 18

The pasteurisation is specifically designed to ensure food safety requirements are achieved in accordance with Australian regulations (Food Standards Australia and New Zealand). As described above, the pasteurisation step is a crucial step in maintaining the native stature of the lactoferrin protein and is designed to minimise the damaging effects of exposure of temperature to the lactoferrin concentrate. The concentrate must undergo heating to 72° C. for a minimum of 15 seconds and the process can be manipulated to ensure the minimum regulatory requirements are achieve whilst maintaining the smallest temperature difference between the heating medium and product temperature. Cooling of the lactoferrin concentrate immediately post this step can help to maintain the bioactivity levels of the lactoferrin.

### 8) Concentrate Storage

Post pasteurisation, the concentrate is stored below 5° C., to accumulate a sufficient volume to process through the fluid bed spray dryer. Ongoing monitoring of the concentrate occurs during this process to ensure the total solids and pH are maintained to the tight limits required to maximise native lactoferrin production.

### 9) Spray Drying 20

The pasteurised lactoferrin concentrate is processed through a multi-stage spray drying process that gently dries the lactoferrin at lower temperatures than conventional spray drying process. The first stage of the drying process is designed to reduce most of the moisture from the concentrate forming the powder particle. This part of the drying process is specifically controlled using airflows, air temperatures and concentrate nozzle pressures to achieved specifically designed powder particles to meet customer needs and specifications. The second stage of drying consists of the drying fluidising bed where lower temperature air and air flowrates are used to gently condition the lactoferrin powder, cooling the protein matrix whilst gently adjusting the moisture content to desired level. The product is then passed through a vibrating sifter to ensure larger particles/foreign matter is removed prior to packaging.

### 10) Packaging

The final stage of the process involves dispensing the lactoferrin powder into specifically designed heavy foil pouches. The packaging material has been carefully selected to ensure that it has excellent barrier properties to ensure maintenance of lactoferrin product integrity for the long shelf life of the product. Packaging formats are typically 5 kg. However, this may vary depending on the customer requirements.

The present invention also provides a lactoferrin powder produced by the process described above, as well as a food product comprising the lactoferrin powder produced by the process. Applications of these products include:
Infant, follow-on and toddler nutrition
Listed, Assessed and registered therapeutic goods and medicines (e.g. capsules, tablets, nasal sprays)
Dietary supplements
Adult nutrition
Sports nutrition
Dairy products (beverages, yoghurts and powders)
Functional foods
Nutritionals and Neutraceuticals

The physical characteristics of the lactoferrin powder produced by the process described above include:
Smaller, more uniform lactoferrin particles permit improved blending and solubility outcomes
Spray dried lactoferrin is more uniform in shape with lighter, fluffier particles and low light scattering effect
Flowability can be varied depending on customer need and desired size
Neutral/bland flavour and non-caking
Heat-sensitive
Spray dried lactoferrin colour (i.e. soft pink) is better suited to dry blending versus freeze dried lactoferrin as its colour “blends” better with base powders

As described above, the inventors have also discovered that the highly functional and biologically active lactoferrin powders described above have therapeutic activity. The experiments described below demonstrate that powdered lactoferrin produced in accordance with the present invention has both an inhibitory effect on virus replication and can stimulate an immune response in vivo and the inventors believe that the results of these experiments enable a reasonable prediction of the therapeutic applications disclosed herein. Indeed, lactoferrin has been suggested as a potential therapeutic agent for use in the treatment or prevention of viral infections, including COVID-19, which is the disease caused by the SARS-COV-2 virus, and the inventors expect that the efficacy of such treatments would be enhanced if lactoferrin powders produced in accordance with the present invention were used, given the advantageous biological and functional properties described above. In effect, the present invention provides lactoferrin in powder form, but which has retained substantially all of the biological activity of native lactoferrin, and which also has beneficial functional properties (e.g. mixing properties, solubility, etc.) for formulating in pharmaceutical applications.

Thus, the present invention provides methods for preventing or treating a viral infection in a patient. In one method, a formulation comprising the lactoferrin powder produced as described above may be administered to the patient (e.g. via the patient's airways). In another method, a formulation comprising the lactoferrin powder produced as described above may be nasally administered to the patient.

In other embodiments and aspects, a formulation comprising the lactoferrin powder produced as described above may be administered to the patient orally (e.g. in a capsule).

Also provided are nasal sprays comprising the lactoferrin powder produced by the process described above, as well as pharmaceutical compositions including the lactoferrin powder produced by the process and a pharmaceutically acceptable excipient.

The inventors envisage that a lactoferrin-based nasal spray may be used up to 4 times daily as a preventative or post exposure prophylaxis against viral infections. Given that lactoferrin is a highly conserved, natural protein that is normally present in mucosal secretions, bovine lactoferrin is unlikely to be immunogenic and should be well tolerated (except for people with dairy allergies). This uniquely positions lactoferrin as a first in class nasal spray-delivered mucosal pan-pathogen inhibitor based on a highly conserved, naturally occurring protein. There are many synthetic virus blocking nasal sprays being developed, but such do not possess the pleiotropic immune boosting functions of lactoferrin.

One particular therapeutic application envisaged by the present inventors is as a potential preventative and adjunct treatment for COVID-19, as well as diseases caused by other viral strains such as those which cause the common cold (Rhinovirus) and influenza. Experiments have therefore been conducted to investigate whether lactoferrin produced in accordance with the present invention may be effective as a therapeutic agent against COVID-19, by examining whether it improves responses to COVID-19 in human bronchial epithelial cell studies and in a human clinical study. These experiments will be described below.

### EXAMPLES

### Example 1—Production of Lactoferrin Powder

The method via which batches of lactoferrin powders in accordance with embodiments of the invention and referred to below (see Example 2) as PnF20224 and PnF21329 will now be described.

Milk was sourced from the Goulburn Valley in Victoria, Australia. Raw whole milk was separated and the skim milk transferred to the lactoferrin plant for processing. The skim milk was maintained at refrigerated temperatures throughout storage before being processed through a Bacterial Removal Separator (Clarifier) that reduces the bacterial load in the raw skim milk and, subsequently, through a series of filters that remove any remaining foreign matter, fat, fat-soluble compounds and insolubles. These filters become increasingly fine as the milk is processed through each, culminating in a 1 μm filter.

Extraction of the lactoferrin from the skim milk was conducted through an ion exchange process, where the skim milk was passed through radial flow columns containing specialised ion-exchange resin. The resin is composed of large, crosslinked agarose beads (100-300 μm) and is tailor-made for industrial applications and the large particle size and physical stability of the base matrix allow the resin to be utilised in high volume commercial extraction processes. The ion-exchange capability of the resin binds the lactoferrin from the skim milk for collection through the elution process.

The bound lactoferrin is eluted from the resin using a series of increasingly concentrated sodium chloride buffer solutions, the highest of which has a concentration of approximately 10% w/v. The lactoferrin eluate is collected and stored for further processing.

Ultrafiltration and diafiltration of the lactoferrin eluate remove sodium chloride ions from the lactoferrin solution and increases the solids in the solution.

Pasteurisation is specifically designed to ensure food safety requirements are achieved in accordance with Australian regulations (Food Standards Australia and New Zealand). The pasteurisation step is designed to minimise the heat profile of the lactoferrin concentrate. To meet regulatory requirements, the concentrate must undergo heating to 72° C. for a minimum of 15 seconds, or equivalent. Cooling of the lactoferrin concentrate immediately post-pasteuriser can help to maintain the bioactivity levels of the lactoferrin.

The lactoferrin concentrate was then processed through a multi-stage spray drying process that gently dries the lactoferrin at lower temperatures than conventional spray drying processes. The first stage of the drying process is designed to remove most of the moisture from the concentrate forming the powder particle. This part of the drying process is specifically controlled using airflows, air temperatures and concentrate nozzle pressures to achieved specifically designed powder particles to meet customer needs and specifications. The second stage of drying consists of the drying fluidising bed where lower temperature air and air flowrates are used to gently condition the lactoferrin powder, cooling the protein matrix whilst gently adjusting the moisture content to the desired level. The product is then passed through a vibrating sifter prior to packing.

The final stage of the process involves dispensing the lactoferrin into specifically designed heavy foil pouches. The lactoferrin powder passed through a metal detector and into the packaging material, which has excellent barrier properties, maintaining PUREnFERRIN product integrity for the long shelf life of the product. Packaging formats are typically 5 kg; however, this may vary depending on the customer requirements.

The parameters of the equipment used in the process described above are set out below in Table 1.

TABLE 1
Process parameters

| Milk characteristics | Unit of Measure | PnF20224 | PnF21329 |
|---|---|---|---|
| Approx. skim milk used | L | 5,690,000 | 925,080 |
| Titratable acidity | — | 0.15 | 0.14 |
| Fat content | % m/v | 0.08 | 0.07 |
| Protein content | % m/v | 3.80 | 3.60 |
| Total solids | % m/v | 9.79 | 9.72 |
| Solids Non-Fat (SNF) | % m/v | 9.65 | 9.47 |
| Lactoferrin columns | Unit of Measure | PnF20224 | PnF21329 |
| Number of columns | — | 3 | 3 |
| Column feed rate | CV/min | 1 | 1 |
| Column feed temperature | ° C. | 50 | 50 |
| Exit cooler temperature | ° C. | 6 | 6 |
| First buffer conductivity | mS/cm | 42.5-43.5 | 42.0-43.0 |
| First buffer volume | CV | 7 | 7 |
| First buffer flow rate | CV/min | 0.25 | 0.25 |
| Second buffer conductivity | mS/cm | ≥102 | ≥102 |
| Second buffer volume | CV | 3 | 3 |
| Second buffer flow rate | CV/min | 0.25 | 0.25 |
| Ultrafiltration (UF) plant | Unit of Measure | PnF20224 | PnF21329 |
| Average brix of lactoferrin concentrate pre- | Bx | 21.0 | 13.5 |
| pasteurisation | | | |
| Average conductivity of lactoferrin | mS/cm | 0.3 | 0.4 |
| concentrate pre-pasteurisation | | | |
| pH adjustment chemical used | | Sodium hydroxide | N/A |
| pH pre-pasteurisation | — | 5.83-6.47 | 5.96 |
| Pasteuriser | Unit of Measure | PnF20224 | PnF21329 |
| Pasteurisation temperature | ° C. | 73-75 | 73-75 |
| Pasteurisation time | seconds | 17.5 | 17.5 |
| Hot water temperature | ° C. | 76-78 | 75-77 |
| Product flow rate | L/hr | 80 | 80 |
| Dryer feed stream characteristics | Unit of Measure | PnF20224 | PnF21329 |
| Temperature during storage | ° C. | 2.3 | <4.0 |
| Volume of concentrate | L | 5,451 | 607 |
| Concentrate feed density | kg/m3 | 1,041.9 | 1036.0 |
| Concentrate feed flowrate | kg/hr | 178.3 | 184.5 |
| Concentrate feed temperature | ° C. | 49.9 | 49.8 |
| High pressure pump feed pressure | bar | 5 | 5 |
| Spray dryer running condition | Unit of Measure | PnF20224 | PnF21329 |
| Spray nozzle combination | Swirl size/Orifice | S4/0.34 | S4/0.34 |
| | size | | |
| Main inlet air flow | kg/hr | 5,146 | 5,037 |
| Main inlet air temperature | ° C. | 175.6 | 189.6 |
| Outlet air temperature | ° C. | 91.4 | 98.4 |
| Nozzle pressure | bar | 170.8 | 190.6 |
| Chamber fluid bed airflow | m³/hr | 240.2 | 252.5 |
| Chamber fluid bed powder temperature | ° C. | 64.9 | 70.7 |
| Chamber fluid bed air temperature | ° C. | 68.3 | 79.9 |
| Fluid bed drying airflow | m³/hr | 180.0 | 186.0 |
| Fluid bed drying air temperature | ° C. | 58.1 | 74.2 |
| Fluid bed cooling airflow | m³/hr | 140.0 | 177 |
| Fluid bed cooling air temperature | ° C. | 41.9 | 44.1 |
| Fluid bed powder discharge temperature | ° C. | 34.3 | 36.9 |
| Fines destination | — | Sifter | Sifter |
| Fluid bed weirs | — | Open | Open |

### Example 2—Comparison of Lactoferrin Powders of the Present Invention with Commercially Available Lactoferrin Powders

The iron saturation levels, identification, quantitation and quality of lactoferrin in a powder form and produced in accordance with the present invention was measured and compared with that of other commercially available lactoferrin powders (see Table 2). The iron saturation levels, identification and quantification using cation exchange chromatography, reversed-phase high performance liquid chromatography (RP-HPLC) and mass spectrometry (MS) of commercial bovine finished lactoferrin powders produced in accordance with the present invention and from different manufacturers are described.

### 1. Materials and Methods

### 1.1. Chemicals

All reagents and chemicals of HPLC or analytical grade were used, including acetonitrile (FSBA955-4), water (FSBW6-4) Optima® LC-MS grade, trifluoroacetic Acid (FSBT/3258/PB05-100 mL) with a purity of 99% and tris-hydrochloride (#BP153-500) were purchased from Fisher Chemicals, Australia. Sodium chloride (#71380-5 kg) was purchased from Sigma-Aldrich, Australia. Sodium hydroxide (#1.06469.1000) was purchased from Merck Millipore, Australia. The bovine lactoferrin standard (lactoferrin from bovine milk 98.10%; Product number: 127-04122; Lot number: CAG5602) was purchased from Novachem, Australia.

### 1.2. Iron and Iron Saturation Analysis

This method is used to determine the percentage iron saturation of lactoferrin by absorbance measurement at 465 nm. The absorbance at 465 nm of a 100% saturated, 10 mg/mL lactoferrin solution=0.48. Based on this fact, the Fe3+, saturation of an unknown solution can be estimated. The solution must be diluted for A280 measurement to give values between 1.00 and 1.50. Solutions must be filtered through a 0.45 μm cellulose acetate filter prior to measurement at A465 and A280. The analysis was conducted by Bureau Veritas Asure Quality (BVAQ) laboratory, North Melbourne, Victoria, Australia, using method GB 5009.268.2-2016 (Iron) and LFST 01 06.03 (Iron saturation).

TABLE 2
Bovine lactoferrin powders analysed

| S. No | Sample description | Manufacturer |
|---|---|---|
| 1 | Bovine lactoferrin powder- | Noumi Limited, AUS |
| | PnF20224 | |
| 2 | Bovine lactoferrin powder- | Noumi Limited, AUS |
| | PnF21329 | |
| 3 | Bovine lactoferrin powder | Leprino Nutrition, US |
| 4 | Bovine lactoferrin powder | Nepean River Dairy, AUS |
| 5 | Bovine lactoferrin powder | Tatura (A Bega Cheese |
| | | Company), AUS |

* = lactoferrin produced in accordance with the present invention

### 1.3. Sample Preparation

### 1.3.1. Lactoferrin Standard and Construction of Calibration Curves Using Fast Performance Liquid Chromatography (FPLC) and Ultra-High Performance Liquid Chromatography (UHPLC)

The lactoferrin reference material for establishing the standard was purchased from FUJIFILM Wako Pure Chemical Corporation (Osaka, Japan) with Product number #127-04122 and Lot number #CAG5602. The stock solution was prepared by dissolving 1 gram of lactoferrin in a 120 ml container with 100 ml of MQ to get 10 mg/ml concentration. The samples were dissolved gently on a Intelli mixer (ELMI RM-2L) at room temperature for 3.5 hours. The working lactoferrin calibration standards were prepared from the stock solution to obtain working standards 0.25, 0.5, 1, 2, 3, 4 and 5 mg/ml.

For UHPLC calibration, a stock solution was prepared at a concentration of 10 mg/ml by dissolving 200 mg of lactoferrin in 20 ml of MQ. Working standards were prepared by dilution of the stock solutions in MQ. The concentrations used for the calibration curve were 0.05, 0.075, 0.1, 0.5, 1, 1.5, 2 and 2.5 mg/ml.

Calibration curves were built for lactoferrin using relative response each target to its respective.

### 1.3.2. Commercial Bovine Lactoferrin Powders Sample Preparation for Cation Exchange Chromatography Analysis Using FPLC

Lactoferrin powders stock solution (10 mg/ml) were prepared by accurately weighing 200 mg of powder in a 50 mL Falcon tube. MQ water added to the tube to a final volume of 20 mL or 20 g. Each powder was prepared in triplicates. The samples were dissolved gently on a Intelli mixer (ELMI RM-2L) at room temperature for 3.5 hours and passed through a 0.45 μm filter. The stock sample was diluted to 2 mg/ml for quantification using cation exchange chromatography (Capto HiRes cation exchange chromatography column, Cytiva).

### 1.3.3. Commercial Lactoferrin Powders Sample Preparation for MS and Callaghan Innovation RP-HPLC Analysis

Lactoferrin powders stock solutions (10 mg/ml) were prepared by accurately weighing 100 mg of powder in a 50 mL Falcon tube. MQ water added to the tube to a final volume of 10 mL or 10 g. Each powder was prepared in triplicates. The samples were dissolved gently on a Intelli mixer (ELMI RM-2L) at room temperature for 3.5 hours and passed through a 0.45 μm filter.

The stock sample was diluted to 1.5 mg/ml for both mass spectrometry and Callaghan Innovation RP-HPLC analysis.

### 1.4. Cation Exchange Chromatography

Cation exchange chromatography experiments were performed at room temperature with an analytical prepacked Capto HiRes S 5/50 column (#29275877, Cytiva Lifesciences) for high resolution separation of native lactoferrin protein using an AKTA pure 25 M2 FPLC system (GE Healthcare, USA). The gradient was followed by protocol supplied by GE Healthcare. The column was regenerated according to the manufacturer instruction after every run.

### 1.5. LC/Q-TOF

The MS and MS/MS analysed were performed using an Agilent Technologies UHPLC 1290 Infinity Quaternary LC system equipped with a diode-array detector (DAD) and Advanced Bio 6545XT LC/Q-TOF instrument. Callaghan RP-HPLC analysis was performed on Aeris™ 3.6 mm WIDEPORE XB-C8 200 Å analytical LC column (Part-OOG-4481-EO, 250×4.6 mm), a C8 Security Guard Ultra Holder (Part-AJO-9000) and Security Guard Ultra Cartridges (Part-AJO-8771) from Phenomenex. A gradient elution was carried out as per Billakanti et al. 2019 (14) with a mixture of two solvents. Solvent A consisted of 0.1% trifluoroacetic acid (TFA) in water and solvent B was 0.1% TFA in acetonitrile. The detection was carried out at the wavelengths of 280 nm. Th injection volume consisted of 20-μl. At least three independent analyses were performed for each sample, and representative results are shown. Agilent Mass Hunter Qualitative Analysis Software 10 was used for data analysis.

The MS analysis by 20-μl injection onto an Agilent Technologies 6545XT Advanced Bio LC/Q-TOF instrument with Dual AJS ESI in positive electrospray ionization mode for the search and identification of lactoferrin intact protein from samples. The ionization source conditions were set as follows: capillary voltage, 4.5 kV; source temperature, drying gas temperature 250° C., drying gas flow 8 l/min, nebulizing gas pressure 2 bar. Collision gas was highly pure nitrogen. All LC-MS measurements were performed in triplicate. Agilent MassHunter BioConfirm 10 software and Sequence Manager software were used for data analyses.

### 2. Results and Discussion

### 2.1. Iron Content and Iron Saturation Levels

The results show that total iron concentration and iron saturation levels are varied in the lactoferrin powders (Table 3). The iron content in the lactoferrin powders was as follows: 105 mg/kg (Leprino Nutrition), 109 mg/kg (PnF20224), 129 mg/kg (PnF21329), 129 mg/kg (Tatura), 147 mg/kg (Nepean River Dairy), and 180 mg/kg (FUJIFILM Wako Pure Chemical Corporation), ranking Nepean River Dairy iron content as highest.

The percentage of iron saturation of the six different lactoferrin powders was calculated based on the ratio of light absorption at 465 nm and 280 nm as measured by UV-Vis Spectroscopy according to BVAQ laboratory. The iron saturation levels ranged from 16% to 23% (Table 3). Typically, lactoferrin in its native form is characterised by iron saturation between 15-20% and has a salmon pink colour. Noumi and Leprino lactoferrin powders all contain 16%, while Tatura is 14%, Nepean River Dairy is 23% (the highest of all analysed) and the lactoferrin reference material from FUJIFILM Wako corporation iron saturation was 19%.

Iron saturation levels affect the physico-chemical properties of the finished lactoferrin powder, which influences the function and efficacy of lactoferrin as lactoferrin function depends on its correct three-dimensional structure. Iron binding promotes lactoferrin changes in its tertiary structure and increases its thermal stability, and native lactoferrin exhibits slightly higher thermal stability than other forms. As the Noumi lactoferrin powders meet the criteria of a salmon pink colour with 16% iron saturation to be called native lactoferrin, it can also be expected to demonstrate slightly higher thermal stability.

TABLE 3
Iron and iron saturation levels in commercial bovine lactoferrin powders

| | | | | | Iron |
|---|---|---|---|---|---|
| | | | | Iron | Saturation |
| S. No | Sample description | Manufacturer | Country | (mg/kg) | (%) |
| 1 | Bovine Lactoferrin powder | Noumi Limited | Australia | 109 | 16 |
| | PnF20224 | | | | |
| 2 | Bovine Lactoferrin powder | Noumi Limited | Australia | 129 | 16 |
| | PnF21329 | | | | |
| 3 | Bovine Lactoferrin powder | Leprino | United | 105 | 16 |
| | | Nutrition | States | | |
| 4 | Bovine Lactoferrin powder | Nepean River | Australia | 147 | 23 |
| | | Dairy | | | |
| 5 | Bovine Lactoferrin powder | Tatura (A Bega | Australia | 129 | 14 |
| | | Cheese | | | |
| | | Company) | | | |
| 6 | Lactoferrin powder, from | FUJIFILM Wako | Japan | 180 | 19 |
| | Bovine Milk | Pure Chemical | | | |
| | | Corporation | | | |

* = lactoferrin produced in accordance with the present invention

### 2.2. Lactoferrin Quantification in Lactoferrin Powders by Cation Exchange Chromatography

A high resolution pre-packed Capto HiRes S 5/50 column was selected for the quantification of lactoferrins to provide superior peak resolution. The results shows that lactoferrin was well separated and eluted for accurate quantification. The chromatograms were recorded at 280 nm.

To determine lactoferrin concentrations, a calibration curve was generated as described in the methods section. The reference lactoferrin produced a good linear calibration curve for the selected concentration range (0.25-5 mg/ml) with R2 value of 0.9997. Following this, bovine lactoferrin powders from 4 different manufacturers were analysed for quantification of lactoferrin content and purity using the optimised assay conditions by GE Healthcare on the Capto HiRes S 5/50 column (Table 4).

The protein purities were determined based on the main lactoferrin peak percentage of the integrated chromatograms. Different lactoferrin purity and quantitation levels were found in the lactoferrin powder samples (Table 3). The protein purities were calculated to be 98.76% and 98.58% for PnF20224 and PnF21329 respectively, 98.36% for lactoferrin (Nepean River Dairy) and 94.84% for lactoferrin (Tatura). The percentage of lactoferrin recovery was 82% and 81% for PnF20224 and PnF21329 respectively, 62% for Nepean River Dairy and 74% for Tatura. The differences in lactoferrin recovery may be ascribed to various purification processes used by the various lactoferrin commercial manufacturers.

TABLE 4
The percentage of lactoferrin recovery in commercial
bovine lactoferrin powders

| | | | Recovered |
|---|---|---|---|
| S. No | Sample description | Manufacturer | LF (%) |
| 1 | Bovine Lactoferrin powder | Noumi Limited | 82 |
| | PnF20224 | | |
| 2 | Bovine Lactoferrin powder | Noumi Limited | 81 |
| | PnF21329 | | |
| 3 | Bovine Lactoferrin powder | Leprino Nutrition | 79 |
| 4 | Bovine Lactoferrin powder | Nepean River Dairy | 62 |
| 5 | Bovine Lactoferrin powder | Tatura (A Bega | 74 |
| | | Cheese Company) | |

### 2.3. Molecular Mass of Lactoferrin in Commercial Lactoferrin Powders

To identify the Molecular Mass of the lactoferrin present in commercial lactoferrin powders, a Q-TOF mass spectrometer was used. The Molecular Mass of intact lactoferrin was measured for PnF20224, PnF21329, Nepean River Dairy and Tatura. The results showed these all aligned with the theoretical Molecular Mass of Lactoferrin: PnF20224 was 83,194 Da, PnF21329 was 82,853 Da, Nepean River Dairy was 83,373 Da and Tatura was 83,984 Da. The mass spectrometry results are presented in Table 5.

Previous research has shown that lactoferrin has been isolated in multiple Molecular Mass forms from bovine colostrum and mature milk, which have been designated as lactoferrin-a (Molecular Mass ˜84,000) and lactoferrin-b (Molecular Mass ˜80,000). These multiple mass forms exist naturally in various milk samples and the different forms are at least partly due to differences in the degree of glycosylation. Noumi's lactoferrin samples (i.e. those of the present invention) are both in the range of 83,000 Da, which indicates that Noumi's lactoferrin is homogenous for the Molecular Mass forms in its samples. Both the Nepean River Dairy (NRD) lactoferrin and Tatura lactoferrin contain more than one Molecular Mass lactoferrin forms (NRD: 83,373 Da-85,595 Da and Tatura: 83,984 Da-88009 Da), indicating a lower quality of lactoferrin.

TABLE 5
Molecular Mass of lactoferrin in commercial
bovine lactoferrin powders

| | | | LF |
|---|---|---|---|
| | | | Molecular |
| S. No | Sample description | Manufacturer | Mass (Da) |
| 1 | Bovine Lactoferrin powder | Noumi Limited | 83194 |
| | PnF20224 | | |
| 2 | Bovine Lactoferrin powder | Noumi Limited | 82853 |
| | PnF21329 | | |
| 3 | Bovine Lactoferrin powder | Nepean River Dairy | 83373 |
| 4 | Bovine Lactoferrin powder | Tatura (A Bega | 83984 |
| | | Cheese Company) | |

### 2.4. Lactoferrin Quantification Using Callaghan Innovation RP-HPLC Analysis

The Callaghan Innovation RP-HPLC analytical method was developed for the simultaneous determination of lactoferrin purity and quantity in commercial lactoferrin products. For the current experiments, the calibration curve was linear over the range of 0.05-2.5 mg/ml with a correlation coefficient (R²) value 1. The lactoferrin content in the tested lactoferrin powders was determined and summarised in Table 6. The lactoferrin recovery for PnF20224 was 81.20%, for PnF21329 was 83.00%, for Nepean River Dairy was 71.37% and for Tatura was 81.79%. Investigation of contaminated proteins in lactoferrin powders is beyond the scope of the present research, but Tatura lactoferrin powder was investigated by Lonnerdal et al. in 2020, who found that along with the ˜80,000 Da major LF protein, a minor lactoferrin fragment ˜25,000 Da was identified, which indicates that lactoferrin was fragmented during the production process.

TABLE 6
The percentage of lactoferrin recovery in commercial bovine
lactoferrin powders using Callaghan RP-HPLC analysis

| | | | Recovered |
|---|---|---|---|
| S. No | Sample description | Manufacturer | LF (%) |
| 1 | Bovine Lactoferrin powder | Noumi Limited | 81.20 |
| | PnF20224 | | |
| 2 | Bovine Lactoferrin powder | Noumi Limited | 83.00 |
| | PnF21329 | | |
| 5 | Bovine Lactoferrin powder | Nepean River Dairy | 71.37 |
| 6 | Bovine Lactoferrin powder | Tatura (A Bega | 81.79 |
| | | Cheese Company) | |

In the current study, the iron content, iron saturation levels, protein purity and quantification (Cation exchange and RP-HPLC) was evaluated for lactoferrin powders from Noumi Limited and two other Australian lactoferrin manufacturers (Nepean River Dairy and Tatura). All lactoferrins showed considerable differences in iron saturation, purity and quantification. Both cation exchange chromatography and RP-HPLC analytical lactoferrin quantification reveal that both analytical techniques have individual benefits in studying the different physiochemical properties to identify lactoferrin quantification. The FPLC, cation exchange chromatography and RP-HPLC results show differences in lactoferrin recovery, where the Noumi lactoferrin (i.e. in accordance with the present invention) has a high recovery. Combining cation exchange and RP-HPLC techniques provides a better understanding of bovine lactoferrin isolated powders: cation exchange analysis provides insights into heat induced changes on lactoferrin under native buffer conditions, whereas RP-HPLC will not provide such information (14, 21). Both chromatography quantification analyses have demonstrated that Noumi lactoferrin powders (PnF20224 and PnF21329) have high recovery and lactoferrin protein is stable during manufacturing conditions, leading to minimise the loss of biological activity and suitable for clinical research, dietary supplements, and nutrient formulas.

### Example 3—In Vitro Investigations of Antiviral Activity

A multi-pronged research approach of conducting a human lung cell study, subsequent in vivo rodent studies and a human clinical study will aim to provide evidence of the therapeutic effects described herein. Unless stated otherwise, references to “Lactoferrin” in Examples 3 and 4 refer to lactoferrin in the form of a lactoferrin-containing powder produced in accordance with the method of the present invention.

Ethics Statements, Donor Recruitment, and pBEC Collection.

Primary bronchial epithelial cells (pBECs) were provided by P. A. B. Wark (The University of Newcastle), obtained from healthy non-smoking donors during bronchoscopy, with written informed consent. All subjects underwent fiber-optic bronchoscopy in accordance with standard guidelines. pBECs were obtained using a single sheathed nylon cytology brush applied under direct vision. Approximately 4-8 brushings were taken from second to third generation bronchi.

Air-Liquid Interface Culture of pBEC and Conditionally Reprogramed pBEC

Culture and differentiation at air-liquid interface was performed according to previously described methods (Loo SL, Wark PAB, Esneau C, Nichol KS, Hsu AC, Bartlett NW. Human coronaviruses 229E and OC43 replicate and induce distinct antiviral responses in differentiated primary human bronchial epithelial cells. Am J Physiol Lung Cell Mol Physiol 2020; 319: L926-L931). Prior to culture at air-liquid interface, pBECs obtained from one asthma donor were grown and differentiated until confluent. For pBECs obtained from healthy patients, cells were conditionally reprogrammed by co-cultured with irradiated fibroblasts according to a previously published protocol (Martinovich KM, Iosifidis T, Buckley AG, Looi K, Ling KM, Sutanto EN, Kicic-Starcevich E, Garratt LW, Shaw NC, Montgomery S, Lannigan FJ, Knight DA, Kicic A, Stick SM. Conditionally reprogrammed primary airway epithelial cells maintain morphology, lineage and disease specific functional characteristics. Sci Rep 2017; 7:17971). After 25-30 days of differentiation the culture was confirmed to be ready by the presence of pseudostratified structure, ciliated epithelium and presence of mucus/mucus-producing cells.

### Virus Stock and Propagation

OC43 virus stock was obtained from ATCC (VR-1558). American Type Culture Collection (ATCC) indicates that OC43 was propagated on HCT-8 cells with a number of passages unknown. The ATCC-supplied OC43 titre was 2.8×105 median tissue culture infectious dose per milliliter (TCID50/mL) and Upon receiving the ATCC stocks, the virus was passaged three times in MRC-5 cells to generate working stocks: OC43 titrewas 2×108 TCID50/mL and used for studies according to WHO guidelines. Viral titre was measured by TCID50 assay in MRC-5, using the Spearman-Karber method.

### Lactoferrin Treatment and Infection

Bovine lactoferrin (LF) powder (Noumi Limited, Australia) was diluted to a stock concentration of 10 mg/mL in PBS. Once fully differentiated, apical treatment with LF was done by the addition of 50 μL of 100 μg/mL (LF100) and 1000 μg/mL (LF1000) diluted in BEBM minimal medium (MM). Differentiated pBECs were infected with OC43 at a moi of 0.1. The virus inoculum was diluted in 200 uL MM, with corresponding concentration of LF to maintain exposure to LF during binding.

Prior to infection, cells were washed once with PBS and pre-treated for 3 hours with 50 μL of LF at corresponding concentration (or MM control) on the apical surface only. After three hours, the virus inoculum was directly added to the well, without removing the LF treatment (total infection volume 250 uL). After 2 hours incubation at 35° C., the inoculum was removed. Cells were washed with 500 ul PBS to remove unbound virus. Finally, 50 μL of LF treatment was added apically, corresponding to the start of the time course. Treatment was maintained during the duration of the time course, and pBECs were harvested at 0, 24, 48 and 96 hr post infection. Where applicable (FIG. 5) 50 μL of LF treatment (or MM control) was added to the apical surface at 48 hours post infection in order to refresh the treatment.

Sample Collection and Analysis from ALI Cultures

Upon harvest, apical washes were collected by addition of 450 uL (or by addition of 400 uL when the LF treatment was refreshed at 48 hours post infection) PBS for 5 minutes to measure the release of infectious virus. Half-membranes were collected in RLT buffer (Qiagen) containing 1% 2-mercaptoethanol (2ME) for molecular analyses and in RIPA buffer containing protease inhibitor cocktail (Roche) for protein analyses. All samples were stored at −80° C. until further use.

### Measure of Released Infectious Virus by TCID50

OC43 working stock titre and released infectious virus in apical washes was measured using TCID endpoint dilution method. MRC-5s were seeded at a concentration of 1.105 cells/well in 10% FCS EMEM media (Hyclone) a flat bottom 96 well plate. The next day, when cells reached 40% confluence, cells were infected with the samples, which were serially diluted in 1% EMEM media. One row was kept as control. Plates were incubated for 5 days at 35° C. and examined using a microscope to determine the presence or absence of CPE in each column. Using these results, infectious viral units were determined using the Spearman-Karber method.

Measure of Viral Load by qPCR

Total RNA was extracted using miRNAeasy mini kit (Qiagen). Purified RNA was quantitated by spectrophotometry (Nanodrop). cDNA was synthesized using the High-Capacity cDNA Reverse Transcription Kit (ABI). Standards were produced for qPCR using plasmids for the OC43 nucleocapsid gene PCR detection. Viral load qPCR analysis was conducted using the ABI 7500 Real Time PCR System, using a taqman assay targeting OC43 N gene (Geneworks). All targets were normalized using 18S (Applied Biosystems, Thermofisher Scientific). Table 7 shows the primers and probes for these two assays.

TABLE 7
qPCR primers and probes

| Target Gene | Reverse Primer | Forward Primer | Probe |
|---|---|---|---|
| N-OC43 | 5′- | 5′- | 5′-FAM- |
| | CGATGAGGCTATTC | CCTTCCTGAGCCTTCAATATA | TCCGCCTGGCACGGTACTCCCT- |
| | CGACTAGGT | GTAACC | TAMRA |
| 18S | 5′- | 5′- | 5′-FAM- |
| | CGCCGCTAGAGGTG | CATTCTTGGCAAATGCTTTCG | ACCGGCGCAAGACGGACCAGA- |
| | AAATTCT | | TAMRA |

### Statistical Analysis

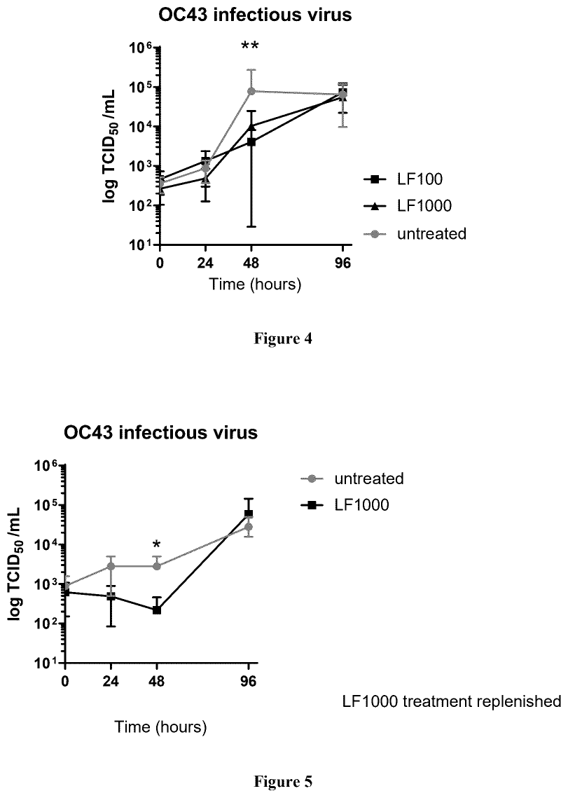

All data was analyzed using GraphPad Prism version 8.2.1 software. qPCR data (FIG. 3) was compared by two-way ANOVA with Šidák multiple comparisons test. Log transformed infectious virus data measured by TCID50 (FIGS. 4 and 5) was compared by two-way ANOVA with Šidák multiple comparisons test. A P value <0.05 was considered statistically significant.

### Results

### Human Differentiated Primary Human Bronchial Epithelial Cell Study

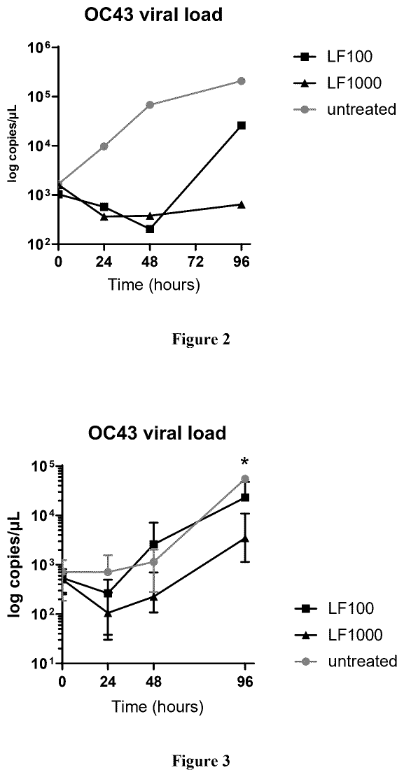

As can be seen in FIG. 2, apical treatment with lactoferrin showed a reduction of OC43 viral load over a 96 hours time course in pBEC that were obtained from one donor with asthma differentiated at air liquid interface. Cells were infected with OC43 at moi of 0.1, in the presence or absence of lactoferrin treatment (LF100: 100 μg/mL; LF1000: 1000 μg/mL) diluted in minimal media (MM). OC43 viral load was measured using an OC43 specific assays targeting the N gene. Data was normalised to 18s housekeeper gene using 18s specific assay. N=1

As demonstrated by FIG. 2, a single treatment with 1000 μg/mL lactoferrin (produced in accordance with the present invention) of differentiated primary human bronchial epithelial cells resulted in inhibition of 100% up to 4 days post infection. The 10-fold lower, 100 μg/mL dose was also effective, showing 100% blockade of virus replication after 2 days, and approx. 90% reduction at 4 days post infection.

As can be seen in FIG. 3, apical treatment with 1000 μg/mL lactoferrin also showed a significant reduction of OC43 viral load at 96 hours post infection. pBECs obtained from two healthy donors were differentiated at air liquid interface. Cells were infected with OC43 at moi of 0.1, in the presence or absence of lactoferrin treatment diluted in minimal media (MM). OC43 viral load was measured using an OC43 specific assays targeting the N gene. Data was normalised to 18s housekeeper gene using 18s specific assay. Statistical analysis was performed using two-way ANOVA with Šidák multiple comparisons test and the error bars represent mean +/−SD; N=8; p<0.05 (* on graph)

Subsequent experiments showed infectious virus being shed at the apical surface of the airway epithelial cultures (FIGS. 4 and 5). As can be seen in FIG. 4, apical treatment with 1000 μg/mL lactoferrin showed a significant reduction of OC43 released infectious virus at 48 hours post infection. pBECs obtained from two healthy donors were differentiated at air liquid interface. Released infectious virus was measured by TCID50 assay using the Spearman-Karber calculation and log transform of data was analysed by two-way ANOVA with Šidákmultiple comparisons test. Cells were infected with OC43 at moi of 0.1, in the presence or absence of lactoferrin treatment diluted in minimal media (MM).

As can be seen in FIG. 5, apical treatment with 1000 μg/mL lactoferrin showed a significant reduction of OC43 released infectious virus at 48 hours post infection. pBECs obtained from one healthy donor were differentiated at air liquid interface. Cells were infected with OC43 at moi of 0.1, in the presence or absence of lactoferrin treatment diluted in minimal media (MM). Released infectious virus was measured by TCID50 assay using the Spearman-Karber calculation and log transform of data was analysed by two-way ANOVA with Šidák multiple comparisons test. Error bars represent mean+/−SD; N-3; p<0.05 (* on graph). These data showed that LF1000 was statistically lower than untreated at 96 h (lactoferrin was replenished at 48 h).

Lactoferrin treatment at time of infection significantly suppressed production of infectious virus by over 2 logs at 48 h. The virus began to catch up in treated cells by 96 h (FIG. 4), but when lactoferrin was replenished at 48 h, it was still statistically lower (LF1000) than in untreated cells (FIG. 5) and there was no immune system help. In vivo, it is expected that lactoferrin will provide a window of protection over the first days of infection whilst stimulating immunity to provide ongoing anti-viral support. The in vivo mouse study (described below) is expected to illustrate this.

Thus, lactoferrin produced in accordance with the present invention reduces infection by human coronavirus (CoV) OC43 in an in vitro model using differentiated primary human bronchial epithelial cells (data is confidential but available for discussion on request). These positive results are so impressive that in vitro and in vivo (i.e., rodent model) studies are being planned to investigate the effects of the COVID-19 causing virus inhibition with novel treatment modalities (lactoferrin applied topically in an animal's nasal cavity), for application as an antiviral medicine (e.g. for COVID-19) and non-prescription over-the-counter medicine.

### Example 4—In Vivo Investigations of Antiviral Activity in Rodents

Many viruses (including beta-coronaviruses such as OC43 and SARS-COV-2) use proteoglycans as a co-receptor to expedite binding to cells and facilitate infection. By binding to these cell surface proteoglycans, native lactoferrin has been shown to interfere with viral infection. Studies in vitro (in cell lines) have reported the anti-viral activity of lactoferrin for a range of respiratory viruses, including SARS-COV-2. The experiments described below were conducted to determine whether the lactoferrin in lactoferrin powders prepared in accordance with the present invention retained the biological activity of native lactoferrin.

As described above, the antiviral properties of lactoferrin have been demonstrated in differentiated primary human bronchial epithelial cells infected with endemic human coronaviruses (e.g., OC43 and 229E). Subsequent experiments have been conducted in order to obtain early proof of concept of the efficacy against SARS-COV2 in this physiologically informative in vitro model of human airway epithelium.

The experiments described below were conducted using procedures which are standard in the art and which are well-known to skilled persons.

### Intranasal Treatment Efficacy Against OC43 Infection in Mice

### Rationale

Antivirals are likely to play a key role in containment of COVID-19, as well as treatment for the many current and future respiratory viral diseases. The anti-microbial/anti-viral activity of native lactoferrin is recognised, proposed to be mediated by binding to sugar structures on the surface of cells (e.g. heparan, sialic acid-based molecules) which are used by many viruses for cell binding during infection. For example, human respiratory viruses such as coronaviruses utilise sugar-based molecular structures for cell binding: SARS-COV-2 binds to heparan sulfate. A related human betacoronavirus OC43 utilises the glycan-based receptor 9-O-acetylated sialic acids.

These experiments will focus on testing the efficacy of intranasally delivered lactoferrin powder produced in accordance with the present invention (for example as described in Example 1) on blocking OC43 infection using a unique mouse coronavirus (OC43) infection model developed in house to provide proof of concept evidence supporting the development of lactoferrin formulations as an anti-respiratory viral nasal spray. As noted previously, unless stated otherwise, references to “Lactoferrin” in this Example are to lactoferrin in the form of a lactoferrin-containing powder produced in accordance with the method of the present invention.

### Objectives

1. To determine if intranasal lactoferrin treatment reduces infection by human coronaviruses (CoV) OC43 in mice.
2. To analyse antiviral effect of lactoferrin and Immune transcriptome biomarkers

### Study Design

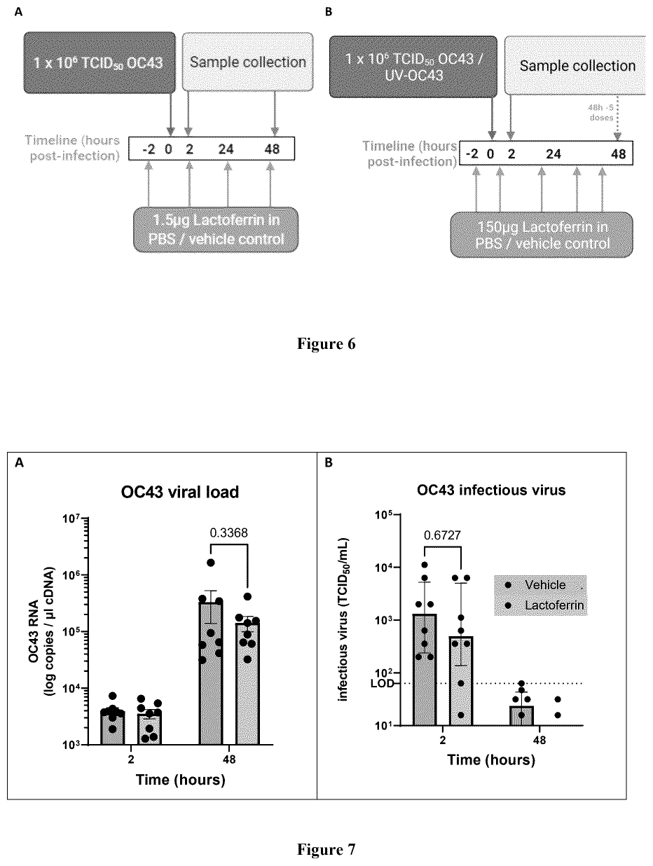

Three separate in vivo experiments were performed to provide proof of concept evidence supporting the development of lactoferrin formulations as an anti-respiratory viral nasal spray. A summary of experimental design for each study is available in Table 8 and FIG. 6Error! Reference source not found.

### Study 1: Proof of Concept for Lactoferrin

Mice (8/group) were treated intranasally with lactoferrin at time of infection and daily after infection (100 μg/mL*15 uL intranasal, corresponding to 1.5 ug total dose per mouse, based on in vitro study) to model post-exposure prophylaxis. Following initial lactoferrin treatment, mice were infected 1×10⁶ TCID50/mL OC43 (or UV-inactivated virus control-UV-OC43) to model the course of an OC43 upper respiratory tract infection, as per established protocol. Experimental endpoints were assessed at 2 and 48 hours post infection. Nasal turbinates and nasal washes were collected for assessment of OC43 viral load by qPCR and infectious by virus median tissue culture infective dose (TCID50), respectively.

### Study 2: Repeat Experiment

Mice (8/group) were treated intranasally with lactoferrin at time of infection and daily after infection (100 μg/mL*15 uL intranasal, corresponding to 1.5 ug total dose per mouse) to model post-exposure prophylaxis. In this repeat experiment, lactoferrin treatment was made fresh each day of the time course. Following initial lactoferrin treatment, mice were infected 1×10⁶ TCID50 OC43/mL (or UV-inactivated virus control-UV-OC43) to model the course of an OC43 upper respiratory tract infection, as per established protocol. Experimental endpoints were assessed at 2 and 48 hours post infection. Nasal turbinates were collected for assessment of OC43 viral load and innate antiviral immune responses (interferons IFN-β and IFN-22/3) by qPCR. Nasal washes were collected for assessment of infectious by virus median tissue culture infective dose (TCID50), respectively.

### Study 3: Immune Transcriptome Biomarker

Mice (8/group) were treated intranasally with lactoferrin at time of infection and twice daily after infection (10 mg/mL*15 uL intranasal, corresponding to 150 ug total dose per mouse) to model post-exposure prophylaxis. Following initial lactoferrin treatment, mice were infected 1×10⁶ TCID50/mL OC43 (or UV-inactivated virus control-UV-OC43) to model the course of an OC43 upper respiratory tract infection, as per established protocol. Experimental endpoints were assessed at 2 and 48 hours post infection. Nasal turbinates were collected for assessment OC43 viral load by qPCR. Broncho-alveolar lavages were collected for leucocyte enumeration. Additionally immune transcriptome biomarkers units in nasal tissue were analysed by Nanostring using the nCounter mouse Immunology v2 Expression panel from Nanostring (Nanostring, Seattle, WA), which assesses the expression of n >500 general mouse immunology-related genes.

TABLE 8
Summary of study design and treatment for each PUREnFERRIN ® in vivo study.

| | | | | Harvest | |
|---|---|---|---|---|---|
| | | Experimental | Administration | (hours post | |
| | Mice 8/group | group | protocol | infection) | Endpoints |
| Study | 2 groups | 1. Vehicle/OC43 | LF or vehicle | 2, 48 | Analysis of viral titres and |
| 1/2 | 16 | 2. 1.5 μg | Day-0 + daily | | interferon gene expression |
| | mice/harvest | Lactoferrin/OC43 | thereafter | | (study 2 only) in nasal tissue |
| | 2 harvests | | | | by PCR. |
| | | | | | Analysis of Live infectious |
| | | | | | viral units in nasal wash by |
| | | | | | TCID50 assay. |

Grand total: 32

| | | | | | |
|---|---|---|---|---|---|
| Study | 4 groups 32 | 1. Vehicle/OC43 | LF or vehicle | 2, 48 | Analysis of viral titres in |
| 3 | mice/harvest | 2. 1.5 μg | Day-0 + twice | | nasal tissue by PCR. |
| | 2 harvests | Lactoferrin/OC43 | daily | | Leucocyte enumeration in |
| | | 3. 150 μg | thereafter | | broncho alveolar lavages |
| | | Lactoferrin/PBS | | | Analysis of immune |
| | | 4. Mock | | | transcriptome biomarkers |
| | | treated/infected | | | units in nasal tissue by |
| | | | | | Nanostring |

Grand total: 64

### Results

### Study 1: Lactoferrin Intranasal Treatment Showed a Trend Toward a Reduction in OC43 Viral Load and Infectious Virus

FIG. 7A shows OC43 viral load in nasal turbinate in untreated (vehicle) mice, versus mice treated with 1.5 ug of lactoferrin intranasally over a 48-hour time course. At 2 hours post-virus inoculation, OC43 viral load was comparable between vehicle and lactoferrin treated mice. By 48 hours of infection, a 2-log increase in OC43 viral load was observed, suggesting viral replication in the upper respiratory tract. For lactoferrin treated mice, there was a trend toward a reduction in viral load at 48 hours post infection, when analysed using 2-way ANOVA statistical test and Sidak multiple comparison test (p=0.3368).

FIG. 7B shows OC43 infectious virus measured in MRC-5 cells using TCID50 assay in nasal wash samples. At 2 hours post virus inoculation, no significant differences in infectious virus doses between the untreated (Vehicle) and LF treated samples were observed, but there was a trend towards a reduction in infectious virus in the lactoferrin treated group (p-0.6727). For the infectious virus TCID50 assay, OC43 live infectious virus at 48 hours post infection fell below the limit of detection (LOD) of the TCID50 assay.

As can be seen in FIG. 7, the OC43 viral load and infectious virus tended to be reduced following lactoferrin intranasal treatment. Mice were treated intranasally with 1.5 ug of lactoferrin at the time of infection and daily thereafter. A) viral load; and B) infectious OC43 levels between vehicle and lactoferrin treated mice were assessed by qPCR and TCID50 assays, respectively. Results were analysed by 2-way ANOVA using Sidak multiple comparison test. N=8; LOD=limit of detection (63.2 TCID50/mL).

Study 2: Lactoferrin Intranasal Treatment Showed a Trend Toward a Reduction in OC43 Viral Load at 48 Hours Post Infection, which was Associated with a Reduction in IFN mRNA.

Since the initial preliminary study showed a trend towards a decreased in OC43 viral RNA and infectious virus, the experiment was repeated. In contrast with the initial experiment, where lactoferrin treatment was made from previously frozen aliquots of a stock lactoferrin concentration, lactoferrin powder provided by Noumi Limited was diluted fresh each day of the treatment.

OC43 viral load and infectious virus tended to be reduced following lactoferrin intranasal treatment. Mice were treated intranasally with 1.5 ug of lactoferrin at the time of infection and daily thereafter. A) viral load; and B) infectious OC43 levels between vehicle and lactoferrin treated mice were assessed by qPCR and TCID50 assays, respectively. Results were analysed by A) 2-way ANOVA using Sidak multiple comparison test and B) Unpaired T test, n=8.

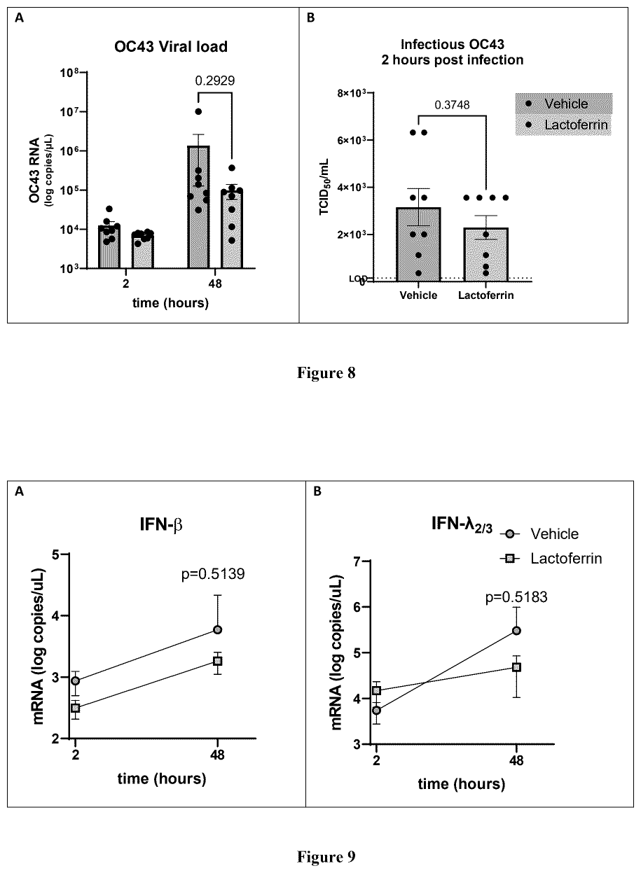

Similar results for OC43 viral load as in the first study were observed. FIG. 8A shows OC43 viral load in in nasal turbinate in untreated (vehicle) mice, versus mice treated with 1.5 ug of lactoferrin intranasally over the 48-hour time course. 2 hours post-virus inoculation, OC43 viral load was comparable between vehicle and LF treated mice. A 2-log increase in OC43 viral load at 48 hours post virus inoculation was observed, suggesting viral replication the upper respiratory tract. There was a trend toward a reduction in viral load at 48 hours post infection in LF treated mice; 2-way ANOVA statistical test and Sidak multiple comparison test (p=0.2929).

Based on results from study 1, OC43 titre was below the limit of detection of the TCID50 assay by 48 h, therefore, live virus was only measured at 2 h post infection. FIG. 8B shows OC43 infectious virus measured in MRC-5 cells using TCID50 assay in nasal wash samples at 2 hours post virus inoculation. A trend towards a reduction in infectious virus in the LF treated group was observed (p=0.3748).

Innate antiviral immune responses in nasal tissues were then investigated using in house IFN-β and IFN-22/3 qPCR assays. Results are presented in FIGS. 9A and 9B, for IFN-β and IFN-22/3, respectively. In FIG. 9, the magnitude of innate immune genes expression after OC43 infection followed viral load. Mice were treated intranasally with 1.5 ug of lactoferrin at the time of infection and daily thereafter. A) IFN-β and B) IFN-22/3 levels between vehicle and lactoferrin treated mice were assessed by qPCR. Results were analysed by 2-way ANOVA using Sidak multiple comparison test. N=8

For both IFN molecules, there was a trend toward a reduction in mRNA levels at 48 hours post infection which followed the reduction in viral load observed at 48 h (Error! Reference source not found.A); analysed using 2-way ANOVA statistical test and Sidak multiple comparison test (p=0.25139 and 0.5183 for IFN-β and IFN-22/3, respectively).

### Study 3: Lactoferrin Intranasal Treatment

In a final study, the antiviral efficacy of a lactoferrin intranasal treatment at a 100× higher dosage and with more frequent dosing was investigated. In this experiment, mice were treated intranasally with 150 ug of lactoferrin, and received 5 intranasal doses in total by the 48-hour post infection time point. Two additional control groups were added in the experimental designs: UV-OC43/untreated (Mock Vehicle) and UV-OC43/LF treated (Mock+LF).

Viral RNA was below the limit of detection in nasal turbinates for groups infected with UV-OC43, indicating that viral RNA levels in the nasal tissue were due to live virus, rather than persistent detection of the initial inoculum. In OC43 infected groups, we observed a one log increase in OC43 viral load, both for vehicle treated and lactoferrin treated groups, between 2 hour and 48 hours of infection. Finally, there was no difference in viral load between vehicle and LF treated group, both at the 2-hour and 48-hour timepoints.

The lactoferrin doses in this study were 100 times higher than those of the earlier studies in order to establish an upper level of tolerance and efficacy. This appears, however, to have been too high, and no effect on viral loading observed. The inventors note that the increased volume of the nasal spray might have simply fallen out of the mice's nasal cavity, for example. Future experiments will only be twice as high (or there abouts).

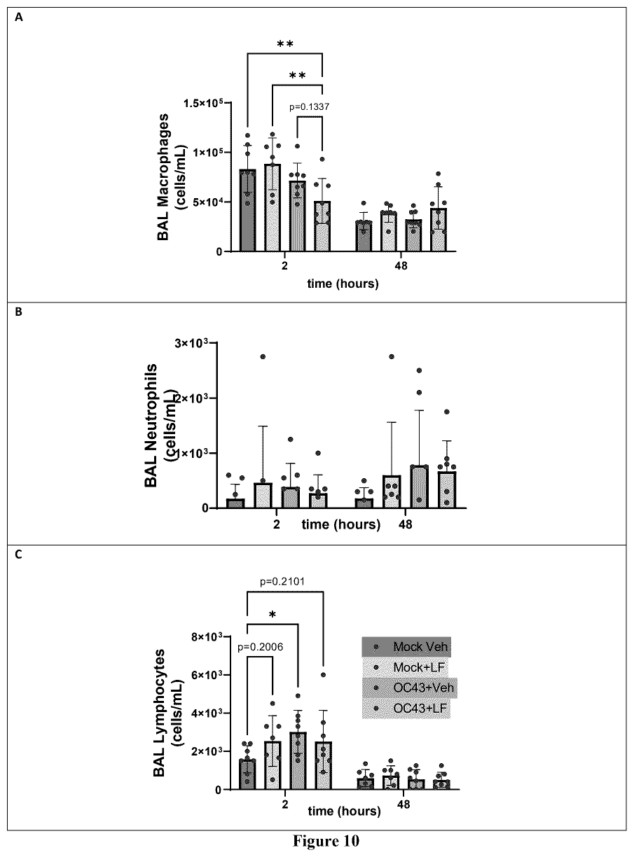

Leucocyte recruitment in broncho-alveolar lavages (BAL) was then investigated to assess the ability of upper respiratory tract lactoferrin treatment to generate protective immune cell recruitment in the lower respiratory tract. As can be seen in FIG. 10, OC43 infection and lactoferrin intranasal treatment. Mice were treated intranasally with 150 ug of lactoferrin at the time of infection and twice daily thereafter and BAL (A) Macrophages, (B) Neutrophils and (C) Lymphocytes were enumerated between vehicle and lactoferrin treated groups and between mock infected and infected groups. Results were analysed in a ‘mixed effect’ statistical test using Tukey's multiple comparison test. n=8 for each group.

FIG. 10A shows the macrophages in the lower respiratory tract. At 2 hours post infection, significant differences between groups were observed. Both groups which were inoculated with UV-inactivated OC43 displayed significantly higher macrophage counts in comparison to groups that were infected with OC43 (p<0.01). Additionally, the OC43 lactoferrin treated group tended to have a lower macrophage count than its untreated counterpart; a mixed effect statistical test using Tukey's multiple comparison (p=0.1337). By 48 hours of infection, there were no differences in macrophage counts within all groups. FIG. 10B shows the BAL neutrophils count at 2 and 48 hours post OC43 inoculation. There was no significant difference in neutrophils count for both timepoints. FIG. 10C shows lymphocytes counts in the lower respiratory tract. At 2 hours post, lymphocytes count in the mock infected untreated group was significantly lower than the OC43 infected vehicle group (p<0.05). There was a trend towards an increase in Lymphocyte for Lactoferrin treated groups compared to the mock infect, vehicle treated group (p=0.2006 and p=2101 for OC43 vehicle and OC43 lactoferrin treated, respectively). By 48 hours of infection, there were no differences in lymphocyte counts.

These data suggest that more regular administrations of lactoferrin may be needed. In humans, 4× day nasal sprays are needed for viral infection prevention effects. Future experiments will treat the mice more frequently, in line with what would occur in typical human treatment regimens.

### Study 3 Continued: Lactoferrin Immune Transcriptome Biomarker

To investigate lactoferrin immunomodulatory activity, the nasal tissues immune transcriptome biomarkers units were analysed using Nanostring mouse Immunology v2 Expression panel (Nanostring, Seattle, WA), which assesses the expression of n>500 general mouse immunology-related genes. Uninfected and untreated groups were compared to an uninfected lactoferrin group and two outliers (one in the vehicle treated group and one in the lactoferrin treated group) were removed for this analysis.

Nanostring revealed upregulation of biomarkers involved in T cell signalling and antigen presentation, lymphocyte recruitment (IL20 CXLC13 and apoptosis (caspase 8), Immune system pathways from macrophages and T-Cell (CD2, CD22, ITG B2), leucocyte migration pathways (ITGB2, ICAM 2 ITGA4. CD2, SELL) and JAK-STAT Signalling pathway (SHP1, STAT), as wells as haematopoietic cell lineage (CD127, HLA-DR, CD19, CD22, CD20).

Data was further analysed for pathways enrichment by lactoferrin treatment. Lactoferrin promoted enrichment of multiple pathways surrounding lymphocyte recruitment, binding, maturation, with antigen presentation and signal transduction pathways (TLR/NFKB/IFN/TNF signalling).

Finally, this analysis revealed that intranasal lactoferrin treatment promoted nasal immune cell recruitment. CD45 (Leucocyte marker), B cells as well as low level neutrophil and T cells were enriched in the LF treated group in comparison to vehicle treated. One exception was cytotoxic t-cell.

### CONCLUSIONS

In this report, the efficacy of intranasally delivered lactoferrin on OC43 infection was investigated using a mouse coronavirus (OC43) infection model developed to provide proof of concept evidence supporting the development of lactoferrin formulations as an anti-respiratory viral nasal spray.

This was done over the span of three in vivo studies, to determine whether intranasal lactoferrin treatment reduced infection by human coronaviruses (CoV) OC43 in mice and perform an analysis of lactoferrin Immune transcriptome biomarkers

Study 1 and 2 focused on the antiviral effect of lactoferrin treatment through investigating viral load and infectious virus between vehicle and mice treated with 1.5 ug of lactoferrin. Importantly, there were strong trends toward a reduction in viral load at 48-hour post infection in both studies.

In the third study, where lactoferrin dosage was increased 100-fold (1.5 ug to 150 ug), viral loads between vehicle treated and OC43 treated group were similar indicating the increased lactoferrin dosage from 1.5 μg to 150 μg was likely too high and failed to effectively prevent infection. Testing additional treatment doses, such as for example 15 μg (i.e. only 10-fold higher), is the next step to determine the optimal dose to prevent OC43 viral load by intranasal lactoferrin treatment

When looking at infectious OC43 in nasal wash samples (study 1 and 2), OC43 binding at 2 hours following inoculation showed a trend towards a reduction in the lactoferrin treated group in comparison to vehicle treated. A further study with increased mice numbers would help provide the statistical power required to reach statistical significance (in addition to treating with lactoferrin earlier and more frequently, as would be the model of treatment in humans). At 48 hours post infection, infectious viral units fell below the limit of detection of the assay. The lack of infectious OC43 by 48 hours post infection, which contrasts with the increase in viral RNA observed by qPCR, is likely an artefact of the experimental model which needs to be optimised further.

Study 3 further focused on the assessment of immune biomarkers following lactoferrin treatment at a higher dose (150 ug per treatment) and with more frequent dosage (twice daily); however, twice daily is still likely too few treatments as in human nasal spray applications, four times daily is typical in terms of application frequency. The aim of study 3 was firstly to determine if upper respiratory tract lactoferrin treatment could generate protective immune cell recruitment in the lower respiratory tract by enumerating leucocytes in broncho-alveolar lavages. At two-hour post infection by OC43 in the upper respiratory tract a significant recruitment of lymphocytes was induced, but not macrophages or neutrophils (p<0.05). At 48 hours post viral challenge, there were no changes in leucocyte counts in the OC43 vehicle treated and OC43 lactoferrin treated groups, in comparison to the UV-OC43 controls, which could indicate that changes in leucocyte counts may be linked to transient perturbations only, or that such effects may be initial immune system ‘priming’ related.

Finally, nasal tissues immune transcriptome biomarkers units were analysed using Nanostring mouse Immunology v2 Expression panel (Nanostring, Seattle, WA) between vehicle treated and LF treated groups. Lactoferrin treatment promoted MHC-II expression, T cell and B cell markers, as well as CXCL13 production, a cytokine which plays an important role in B and T-cell homing. This result suggests that these could be suitable biomarkers in nasal brushings. Pathway enrichment analysis revealed that lactoferrin intranasal treatment promotes enrichment of multiple pathways surrounding lymphocyte recruitment, binding, maturation, with antigen presentation and signal transduction pathways (TLR/NFKB/IFN/TNF signalling). This was further complemented by enrichment cores for immune cell populations such as B and T cells, neutrophils and enriched CD45 (leukocyte marker).

### Methods

### OC43 Propagation, Purification, and Quantification

Original OC43 (ATCC Number VR-1558) passage history is unknown, propagated on HCT-8 cells. Initial stocks from ATCC were received at concentrations of 2.8×105 TCID50/ml and passaged 3 times in MRC-5 cells to generate a working stock as previously described (7). MRC-5 cell lines were used for TCID50 assays using the Spearman-Karber method to quantify viral load.

### Viral Challenge and Treatment Administration

6-8-week-old female BALB/c mice were obtained from Australian Bioresources (Moss Vale, Sydney, NSW). Administration of therapeutics and viral challenge were performed under light anaesthesia using isoflurane. Mice were intranasally challenged with OC43 at a concentration of 1.15×106 TCID50 in 15 uL (study 1 and 2) or 10 uL PBS to model an upper respiratory tract infection. UV-OC43 used in study 3 was inactivated as previously described for respiratory viruses.

### Nasal Lavage, Bronchoalveolar Lavage, and Cell Analysis

Mouse tracheas were cannulated, and the upper respiratory tract was lavaged with HBSS (HyClone, GE Life Sciences) collecting the nasal lavage fluid at the nares. Nasal lavage fluid was stored at −80° C. Bronchoalveolar lavage was processed as previously described for enumeration of leukocytes and cytokines.

### RNA Extraction and Reverse Transcription

Nasal turbinates were excised, and vortexed for 30 seconds in RLT containing 1% betamercaptoethanol. Nasal turbinate debris was removed, and the lysate stored at −80° C. RNA was extracted using the miRNAeasy kit (Qiagen) following the supplier's protocol. RNA was measured by spectrophotometry (Nanodrop) and reverse transcription reaction was performed using 500 ng of RNA with High-Capacity cDNA Reverse Transcription Kit (ABI) per manufacturers' recommendations.

### Quantitative PCR

Quantitative PCR (qPCR) were performed on the QuantStudio 6 using TaqMan Gene Expression Master Mix (Thermo Fisher Scientific) and primer-probe combinations (Thermo Fisher Scientific) as outlined (Table 9). Standards of known concentration were used for absolute quantification of genes of interest. 18s was used as the reference gene to normalize the copy numbers of the genes of interest.

TABLE 9
Table of qPCR primer/probe sequences. Primers and probes were all used
at a final concentration of 5 μM. TaqMan probes all contained 5′FAM
reporter and 3′TAMRA quencher.

| Gene | Primer | sequence |
|---|---|---|
| OC43 | FWD | 5′CGATGAGGCTATTCCGACTAGGT |
| | REV | 5′CCTTCCTGAGCCTTAATATAGTAACC |
| | Probe | 5′FAM-TCCGCCTGGCACGGTACTCCCT-TAMRA |
| IFN-β | FWD | 5′CGCCGCATTGACCATCTA |
| | REV | 5′TTAGCCAGGAGGTTCTCAACAATAGTCTCA |
| | Probe | 5′FAM-TCAGACAAGATTCATCTAGCACTGGCTGGA-TAMRA |
| IFN-λ 2/3 | FWD | 5′AAAGGATTGCCACATTGCTC |
| | REV | 5′TCAAGCAGCCTCTTCTCGAT |
| | Probe | 5′FAM-TCCCCAAAAGAGCTGCAGGC-TAMRA |
| 18s | FWD | 5′CGCCGCTAGAGGTGAAATTCT |
| | REV | 5′CATTCTTGGCAAATGCTTTCG |
| | Probe | 5′FAM-ACCGGCGCAAGACGGACCAGA-TAMRA |

### Virus Median Tissue Culture Infective Dose (TCID50) Virus Infectivity Assay

TCID50 assay for OC43 coronavirus was be carried out in MRC-5 cells using 10% FCS EMEM growth media and 1% FCS EMEM assay media. MRC-5 cells were resuspended in growth media and seeded in 96-well plates (1×105 cells per 100 μL per well). At 30-40% confluence (usually after overnight incubation), cells were infected with nasal wash samples starting at a 2-fold dilution, followed by 10-fold dilution series in EMEM containing 1% FCS. After incubation for 5 days at at 33° C./5% CO2, plates were examined by light microscopy for cytopathological effect. Viral titre for each sample was then determined using the Spearman-Karber method.

### Immune Transcriptome Expression Analysis

Nasal turbinate RNA was hybridized to the nCounter mouse immunology panel (NanoString) and were processed on the Nanostring Prep-station as per manufacturers' instructions with settings for high-sensitivity binding. 555 fields of view were counted on the nCounter Digital Analyzer. Raw data was quality control (QC) checked and normalized to positive/negative controls as well as housekeeper gene expression in the nSolver Analysis software 4.0 for content-normalization QC. Raw counts were imported into nSolver Advanced Analysis software (v2.0.134) for automated normalization using GENorm software and pathways/cell enrichment analyses. The advanced analysis platform used linear regression models to identify DEGs. DEGs were volcano plotted in GraphPad Prism 9.1.2. Heatmapper software was used to generate average Euclidean clustering of samples and gene expression patters as well as visual representations of pathway enrichment z-scores and cell enrichment profiles based on the Nanostring advanced analyses outputs.

It will be understood to persons skilled in the art of the invention that many modifications may be made without departing from the spirit and scope of the invention.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-24. (canceled)

25. A process for producing a lactoferrin powder, the process comprising:
extracting lactoferrin from a native lactoferrin-containing substance and concentrating the extracted lactoferrin to produce a liquid concentrate in which the extracted lactoferrin has an improved stability, wherein the liquid concentrate has a pH of between 5.2 and 7.2 and a concentration of lactoferrin of between about 9 to 12 wt %;
heating the liquid concentrate to a temperature and for a time effective to pasteurize the liquid concentrate; and
drying the pasteurized liquid concentrate under non-denaturing conditions to produce a lactoferrin powder in which substantially all of the lactoferrin is native lactoferrin.

26. The process of claim 25, wherein the extracted lactoferrin has an improved stability because of the absence in the liquid concentrate of species that destabilise the lactoferrin.

27. The process of claim 25, wherein the extracted lactoferrin has an improved stability because of the presence in the liquid concentrate of species that stabilise the lactoferrin.

28. The process of claim 25, wherein the liquid concentrate comprises one or more of the following: a pH adjusting agent, a stabilising mineral and a dissolved gas.

29. The process of claim 25, wherein the native lactoferrin-containing substance is skim milk.

30. The process of claim 29, wherein the skim milk is produced using a cold bowl fat separation process.

31. The process of claim 25, wherein the lactoferrin is extracted from the native lactoferrin-containing substance using ion exchange chromatography.

32. The process of claim 31, wherein a two-step elution is conducted in the ion exchange chromatography, a first elution step comprising eluting species immobilised on the ion exchange column using a buffer solution having a relatively lower concentration, and second elution step comprising eluting lactoferrin immobilised on the ion exchange column using a buffer solution having a relatively higher concentration.

33. The process of claim 25, wherein the extracted lactoferrin is concentrated using ultrafiltration.

34. The process of claim 25, wherein the pasteurized liquid concentrate is cooled immediately after pasteurisation.

35. The process of claim 25, wherein the pasteurized liquid concentrate is dried by spray drying.

36. The process of claim 35, wherein the spray drying comprises multiple stage spray drying.

37. The process of claim 25, wherein in excess of 90% of the lactoferrin in the lactoferrin powder is native lactoferrin.

38. The process of claim 25, wherein the lactoferrin powder consists essentially of lactoferrin.

39. The process of claim 25, wherein the lactoferrin powder has a D50 particle size of about 50 micron.

40. A food product or nasal spray comprising the lactoferrin powder produced by the process of claim 25.

41. A pharmaceutical composition comprising the lactoferrin powder produced by the process of claim 25 and a pharmaceutically acceptable excipient.

42. A method for preventing or treating a viral infection in a patient, the method comprising administering to the patient a formulation comprising the lactoferrin powder produced by the process of claim 25.

43. A method for preventing or treating a viral infection in a patient, the method comprising nasally administering to the patient a formulation comprising the lactoferrin powder produced by the process of claim 25.

44. The method of claim 42, wherein the formulation is administered prophylactically in order to prevent the viral infection.
